(19) United States
(12) Patent Application Publication
(10) Pub. No.: US 2021/0024883 A1
(43) Pub. Date: Jan. 28, 2021

(54) EMULSIONS, METHODS AND USES THEREOF

(71) Applicant: Agency for Science, Technology and Research, Singapore (SG)

(72) Inventors: Jian Li, Singapore (SG); William Richard Nicholas Birch, Singapore (SG); Jessica Pei Wen Toh, Singapore (SG)

(73) Assignee: Agency for Science, Technology and Research, Singapore (SG)

(21) Appl. No.: 16/647,137

(22) PCT Filed: Sep. 14, 2018
(86) PCT No.: PCT/SG2018/050472

§ 371 Date: Mar. 13, 2020

(30) Foreign Application Priority Data
Sep. 14, 2017 (SG) 10201707556P

(51) Int. Cl. C12N 5/00; C08J 3/09; B01J 13/08

(52) U.S. Cl. CPC C12N 5/0075; B01J 13/08; C08J 3/093

(57) ABSTRACT

The present disclosure relates to emulsions, methods of preparation thereof, and uses of said emulsions to fabricate porous polymeric microspheres as microcarriers for cell culture. In particular, the present disclosure relates to an emulsion with enhanced stability, characterized in that the emulsion comprises a) a water phase, the water phase is an aqueous solution comprising a salt; and b) an oil phase, the oil phase comprising a polymer; wherein the oil phase is immiscible with the water phase, and wherein the density differential of the water phase and oil phase is less than about 0.02 g/cm3. In a preferred embodiment, the polymer is polycaprolactone (PCL).

### FIELD

The present disclosure relates to emulsions, methods and uses of said emulsion. In particular, the present disclosure relates to emulsions with enhanced stability and their preparation methods, as well as methods of fabrication of porous polymeric microspheres using certain emulsions disclosed herein.

### BACKGROUND

Water-in-oil (w/o) emulsions are less common than their oil-in-water (o/w) counterparts and thus there are fewer studies describing them. Stability is a very important parameter for an emulsion and often serves in generating more complex systems such as double emulsions. W/o emulsions are often destabilized by coalescence and sedimentation.

The majority of w/o emulsions are systems with an oil phase, having density <1.0 g/cm³, which typically represents hydrocarbon alkane molecules, and their derivatives. Given that it is difficult to lower the density of the aqueous phase, emulsion stability generally relies on the formation of smaller droplets, with a correspondingly reduced weight and higher Brownian motion. This may be complemented by implementing emulsifiers, which stabilize the w/o interface and thus mitigate a primary driver of coalescence.

Porous polymer microspheres can be generated or fabricated by w/o/w double emulsions. Notwithstanding that the resulting microspheres present a wide size distribution, a major challenge is that these w/o emulsions (and correspondingly the resultant double emulsions) are unstable. More recently, quasi-monodisperse droplets have been formed by microfluidics. These droplets subsequently solidify into polymer porous microspheres of uniform size, ranging from microns to hundreds of microns. The instability of w/o emulsions engenders significant challenges in the formation of stable w/o/w emulsions (or droplets) for microfluidic applications.

To get around the problem of emulsion instability, emulsifiers are commonly used. In this regard, emulsifiers reduce the surface tension of the water-oil interface, and act as a physical barrier to contact and coalescence. Alternatively, the emulsifiers can have a charge such that the droplets are electrostatically stabilised. Accordingly, the droplets in the emulsion is prevented from coming together and phase separating. However, a problem with using emulsifiers is that the reduced interfacial tension depends on the concentration of the surfactant according to the Gibbs' isotherm. In this regard, a large amount of emulsifier is required to stabilise an emulsion, which may not be ideal. For example, the cost of production is increased as raw material cost is increased. Further, processing time is also increased as these emulsifiers, being favourably partitioned in both water and oil, are often difficult to remove completely. Such use of emulsifiers may also be disadvantageous in cosmetic formulations as the beauty trend in recent years is towards natural and organic products.

It is generally desirable to develop a stable emulsion that does not phase separate over a desired time frame. It is also desirable to develop a stable emulsion without resorting to the implementation of the use of emulsifiers, which often only serve to delay the coalescence of gravitationally aggregated droplets and add to the cost and process times of the emulsions.

It is generally desirable to overcome or ameliorate one or more of the above mentioned difficulties.

### SUMMARY OF THE INVENTION

In an aspect, the present invention provides an emulsion comprising:
a) a water phase (w); and
b) an oil phase (o);

wherein the oil phase is a solution comprising polymers, oils and/or organic solvents; and

wherein the water phase is an aqueous solution; and

wherein the densities of the water phase and the oil phase are substantially similar

In an embodiment, the emulsion comprises:
a) a water phase, the water phase is an aqueous solution comprising a salt; and
b) an oil phase, the oil phase comprising a polymer;

wherein the oil phase is immiscible with the water phase, and

wherein the density differential of the water phase and oil phase is less than about 0.02 g/cm³.

In some embodiments, the densities of the water phase and oil phase are both >1.0 g/cm³.

In other embodiments, the emulsion is stable for at least 5 days.

In other embodiments, the emulsion does not comprise an emulsifier.

In an aspect, the present invention provides a method of making an emulsion, said method comprising mixing a water phase and an oil phase with substantially similar densities.

In some embodiments, the method of making an emulsion comprises the steps of:
a) homogenising a water phase and an oil phase to form an emulsion, the water phase is an aqueous solution comprising a salt, the oil phase comprising a polymer;

wherein the oil phase is immiscible with the water phase, and

wherein the density differential of the water phase and oil phase is less than about 0.02 g/cm³.

In another aspect, the present invention provides a method of forming a polymeric microsphere, comprising the steps of:
a) homogenising a water phase and an oil phase to form an emulsion, wherein the water phase is an aqueous solution comprising a salt, wherein the oil phase comprising a polymer, wherein the oil phase is immiscible with the water phase, and wherein the density differential of the water phase and oil phase is less than about 0.02 g/cm³;
b) flowing the emulsion into an outer continuous phase to form a double emulsion, the outer continuous phase comprising a dispersant from about 0.1 wt % to about 20 wt % of the outer continuous phase and an emulsifier from about 0.1 wt % to about 20 wt % of the outer continuous phase; and
c) immersing the double emulsion in a solvent exchange liquid to form the polymeric microsphere.

In another aspect, the present invention provides a polymeric microsphere obtained according to the methods disclosed herein.

In another aspect, the present invention provides a polymeric microsphere obtained according to the methods disclosed, the microsphere having a porosity of about 10% to about 90% and wherein the variance of the microsphere particle size distribution is less than about 0.8.

### BRIEF DESCRIPTION OF THE DRAWINGS

Some embodiments of the present invention will now be described by way of non-limiting example only, with reference to the accompanying drawings in which:

FIG. 1 illustrates a graph demonstrating the densities of crude oils as a function of temperature.

FIG. 2 illustrates a graph demonstrating the densities of fuel oils as a function of temperature.

FIG. 3 illustrates a schematic of the production method of a w₁/o emulsion according to the present invention.

FIG. 4 illustrates a schematic of the production method of porous polycaprolactone (PCL) microspheres by using w₁/o/w₂ emulsions from w₁/o emulsion according to the present invention.

FIG. 5 illustrates SEM images of uniform-sized porous PCL microspheres from w₁/o/w₂ droplets formed by microfluidics.

FIG. 6 illustrates a bottle test experiment demonstrating the stability, phase separation and upward floating of water droplets in water-in-oil (w₁/o) emulsions at different time intervals of sample preparation.

FIG. 7 illustrates the average size and size distribution of some examples of water-in-oil (w/o) emulsions 2 hr after the emulsions are prepared.

FIG. 8 illustrates SEM images of cross-section of porous PCL microspheres from w₁/o/w₂ double emulsions.

FIG. 9 illustrates an example of the distribution of microsphere particle sizes made using the method disclosed herein.

FIG. 10 illustrates a bottle test experiment showing the stability, phase separation and upward floating of water droplets in water-in-oil (w₁/o) emulsions at different time intervals of sample preparation.

FIG. 11 illustrates photographs of hMSCs performance on porous PCL45k microspheres with a density of 1.035 g/cm³ under agitation culture.

FIG. 12 illustrates hMSC performance on pPCL45k MCs with low and high density under agitation culture.

FIG. 13 illustrates a flow cytometry analysis of hMSC growth on porous microspheres, with expression of MSC markers CD34, CD73, CD90, and CD105.

### DETAILED DESCRIPTION

“Hydrophilic,” as used herein, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the compound may be considered to be hydrophilic.

“Hydrophobic,” as used herein, refers to molecules which have a greater affinity for, and thus solubility in, organic solvents as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert--butyl ether. If after equilibration a greater concentration of the compound is present in the organic solvent than in the water, then the compound may be considered to be hydrophobic.

The terms “hydrophilic” and “hydrophobic” are relative terms. Hydrophilicity and/or hydrophobicity are determined in any manner suitable. For example, one method of determining the hydrophilicity and/or hydrophobicity of non-ionic amphiphilic compounds is the hydrophilic-lipophilic balance (the “HLB” value). Surfactants with lower HLB values are more hydrophobic/lipophilic, and have greater solubility in oils, whereas surfactants with higher HLB values are more hydrophilic, and have greater solubility in aqueous medium. Using HI,B values as a rough guide, hydrophilic surfactants are generally considered to be those compounds having an HLB value greater than about 10, as well as non-ionic, anionic, cationic, or zwitterionic compounds for which the HLB scale is not generally applicable. Similarly, lipophilic surfactants are compounds having an HLB value less than about 10.

“Water phase” as used herein, refers to a water based solvent or solvent system, which comprises of mainly water. Such solvents can be either polar or non-polar, and/or either protic or aprotic. Solvent systems refer to combinations of solvents which result in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Water based solvent or solvent systems can also include dissolved ions, salts and molecules such as amino acids, proteins, sugars and phospholipids. Such salts may be, but not limited to, sodium chloride, potassium chloride, potassium carbonate, ammonium acetate, magnesium acetate, magnesium chloride, magnesium sulfate, potassium acetate, potassium chloride, sodium acetate, sodium citrate, zinc chloride, HEPES sodium, calcium chloride, ferric nitrate, sodium bicarbonate, potassium phosphate and sodium phosphate. As such, biological fluids, physiological solutions and culture medium also fall within this definition. In relation to the present invention, the water phase is an aqueous solution comprising at least a salt.

“Oil phase” as used herein, refers to an organic based solvent or solvent system, and which comprises of mainly organic solvent. Organic based solvents can be any carbon based solvents. Such solvents can be either polar or non-polar, and/or either protic or aprotic. Solvent systems refer to combinations of solvents which resulting in a final single phase. Both ‘solvents’ and ‘solvent systems’ can include, and is not limited to, pentane, cyclopentane, hexane, cyclohexane, benzene, toluene, dioxane, chloroform, diethylether, dichloromethane, tetrahydrofuran, ethyl acetate, acetone, dimethylformamide, acetonitrile, dimethyl sulfoxide, nitromethane, propylene carbonate, formic acid, butanol, isopropanol, propanol, ethanol, methanol, acetic acid, ethylene glycol, diethylene glycol or water. Organic based solvents or solvent systems can include, but not limited to, any non-polar liquid which can be hydrophobic and/or lipophilic. As such, oils such as animal oil, vegetable oil, petrochemical oil, mineral oil and other synthetic oils (for example silicone oil) are also included within this definition.

“Emulsion” as used herein, is used in the broadest sense and can refer to either oil suspended in an aqueous phase (o/w), or water suspended in oil (w/o). Milk is an example of an o/w emulsion, in which the fat phase or cream forms tiny droplets within the water phase. The emulsion can further include a further, but not limited to, third phase, such that a w/o/w or o/w/o emulsion is formed. Emulsions also include macroemulsions, microemulsions and nanoemulsions within its definition. A person skilled in the art would understand that macroemulsions usually appear cloudy, have high interfacial tension, are thermodynamically unstable, are at least biphasic, and usually require an input of high shear energy, for their formation. Microemulsions are usually transparent or translucent, have very low interfacial tension, require a large amount of surfactant, are thermodynamically stable, are mono-phasic, and require relatively lower input of energy, for their formation. Nanoemulsions are usually transparent or translucent, requires a large amount of surfactant, and are generally kinetically stable.

As used herein, ‘salt’ refers to an ionic compound that can be formed by the neutralization reaction of an acid and a base. Salts are composed of related numbers of cations (positively charged ions) and anions (negative ions) so that the product is electrically neutral (without a net charge). These component ions can be inorganic, such as chloride (Cl⁻), or organic, such as acetate (CH₃CO₂⁻); and can be monatomic, such as fluoride (F⁻), or polyatomic, such as sulfate (SO₄²⁻).

As used herein, ‘alkali’ refers to a base that is dissolvable in water. In this regard, the alkali in water will form a solution with a pH greater than 7.

As used herein, ‘acid’ refers to a molecule that is capable of donating a proton. In this regard, the acid is Bronsted acid. In water, the acid will form a solution with a pH smaller than 7.

As used herein, ‘sugar’ refers to water-soluble carbohydrates, such as monosaccharides, disaccharides, or oligosaccharides.

As used herein, ‘cyclodextrin’ refers to compounds made up of sugar molecules bound together in a ring. Cyclodextrins are composed of 5 or more α-D-glucopyranoside units linked 1->4, as in amylose (a fragment of starch). Cyclodextrins contain a number of glucose monomers ranging from six to eight units in a ring, creating a cone shape. For example, α (alpha)-cyclodextrin is a 6-membered sugar ring molecule, β (beta)-cyclodextrin is a 7-membered sugar ring molecule, and γ (gamma)-cyclodextrin is a 8-membered sugar ring molecule.

This present disclosure describes water-in-oil emulsions, oil-in-water emulsions, oil-in-water-in-oil double emulsions and water-in-oil-in-water (w₁/o/w₂) double emulsions (also referred to as droplets), and methods of fabricating and stabilizing these emulsions. In particular, the present disclosure describes emulsions that are formable when the density of the water phase and the density of the oil phase are substantially matched. In particular, these emulsions can be macroemulsions. This emulsion is found to be stable (or homogenous) for over long periods of time, for example, for at least 5 days. Preferentially, the present disclosure describes w/o macroemulsions and water-in-oil-in-water (w₁/o/w₂) double emulsions.

The emulsion referred to herein can also be thought of as a composition, the composition comprising of a water phase and an oil phase. Specifically, the water phase can be an aqueous solution of a salt and/or other components are described herein, and the oil phase comprises a polymer. The oil phase is immiscible with the water phase. As is disclosed herein, similar to the emulsion, the composition preferentially has a water phase and oil phase, wherein the density differential of the water phase and oil phase is less than about 0.02 g/cm³.

Without wanting to be bound by theory, the inventors believe that the DLVO (Boris Derjaguin and Lev Landau, Evert Verwey and Theodor Overbeek) theory can be used to rationalise the formation of a stable macroemulsion. The skilled person would know that macroemulsions are not thermodynamically stable. This means that from the moment they are created, they will tend to revert to their original, immiscible and separate state. Macroemulsions can exist because they are kinetically stable rather than thermodynamically stable. The DVLO theory postulates that the stability of colloidal particles is attributed to replusive electrostatic interactions and attractive Van der Waals interactions. The combination of these two forces creates a primary energy minimum (when colloidal particles are irreversibly aggregated) and a shallower secondary energy minimum (when colloidal particles are separated). Separating these energy minimums is an energy barrier that colloidal particles need to overcome to transit to an aggregated state. As is known in the art, surfactants and emulsifiers can be used to further increase the energy barrier. This is due to the surfactants and emulsifier being at the interface of the particles, which reduces the interfacial area and accordingly requiring more energy for the particles to aggregate. Without wishing to be bound by any particular theory, the inventors postulate that an emulsion can be favourable maintained in its desired state if the shallow secondary energy well is instead made deeper. In this regard, the inventors have found that by formulating emulsions with an aqueous phase that is density-matched to the immiscible oil phase, the kinetic stability of the particles in the emulsion (and hence in the secondary energy well) can be improved/increased. This may be partially due to the reduction of creaming, which consequently reduces the probability of coalescence and demulsification.

In this regard, it is believed that the emulsions are advantageously stabilised against coalescence, and hence do not phase separate. The emulsions are also stabilised against gravitational sedimentation. Advantageously, this results in a better kinetically stabilised emulsion.

In an embodiment, the present invention provides an emulsion comprising a water phase (w) and an oil phase (o). The oil phase is a solution comprising oils and/or organic solvents and the water phase is an aqueous solution. The densities of the water phase and the oil phase are substantially similar

In another aspect, the present disclosure discloses an emulsion comprising a water phase (w); and an oil phase (o). The water phase (w₁) can be an aqueous solution. The water phase can further be an aqueous salt solution. The oil phase can be a solution comprising at least a polymer. The oil phase is immiscible with the water phase.

The emulsion is either a water-in-oil (w/o) emulsion or an oil-in-water (o/w) emulsion. In some embodiment, the emulsion is a water-in-oil (w/o) emulsion. In another embodiment, the emulsion is an oil-in-water (o/w) emulsion. In this regard, the water phase and oil phase are substantially immiscible. In a water-in-oil (w/o) emulsion, the water forms an inner water phase, which can be an aqueous solution. The inner water phase can further be an aqueous salt solution. The oil phase forms an outer oil phase, which can be a solution comprising polymers, oils and/or organic solvents. The inverse applies for an oil-in-water (o/w) emulsion; i.e. inner oil phase and outer water phase.

The densities of the water phase and the oil phase are substantially similar. As exemplified in the examples, when the oil phase has a density of about 1.326 g/cm³, to form an emulsion of the present invention, the water phase has a density of about 1.27 g/cm³ to about 1.414 g/cm³. When the oil phase has a density of about 1.06 g/cm³, the water phase has a density of about 1.061 g/cm³. When the oil phase has a density of about 1.09 g/cm³, the water phase has a density of about 1.086 g/cm³. In this regard, the difference in the densities of the water phase and oil phase is less than about 2%, less than about 1.5%, less than about 1%, less than about 0.8%, less than about 0.6% or less than about 0.4%. Alternatively, the density differential (or difference in densities) of the water phase and oil phase is less than about 0.02 g/cm³, less than about 0.018 g/cm³, less than about 0.016 g/cm³, less than about 0.014 g/cm³, less than about 0.012 g/cm³, less than about 0.01 g/cm³, less than about 0.009 g/cm³, less than about 0.008 g/cm³, less than about 0.007 g/cm³, less than about 0.006 g/cm³, less than about 0.005 g/cm³, less than about 0.004 g/cm³ or less than about 0.003 g/cm³. In particular, the density differential is less than about less than about 0.006 g/cm³.

By varying, for example, the salt concentration in the water phase, the density of the aqueous salt solution can be adjusted to be equal to, or close to, the density of, for example, the polymer solution of the oil phase. Therefore, the gravitational stabilization of w/o emulsions (or o/w emulsions) is realized by matching the density of the aqueous salt solution with the density of the polymer solution (oil phase).

For example, the w/o emulsions may have a water phase with a density of 0.95-1.05 g/cm³, and an oil phase with density <1.0 g/cm³, for example, animal, vegetable or mineral oil. Some organic materials and/or organic solvents with density >1.05 g/cm³ can be add to mix into the oil phase to make the density to increase to be 0.95-1.05 g/cm³, which matches the density of the water phase.

In some embodiments, the densities of the water phase and oil phase are independently from about 1 g/cm³ to about 1.7 g/cm³. In other embodiment, the densities are independently from about 1.310 g/cm³ to about 1.335 g/cm³, about 1.312 g/cm³ to about 1.334 g/cm³, about 1.314 g/cm³ to about 1.332 g/cm³, about 1.316 g/cm³ to about 1.332 g/cm³, about 1.318 g/cm³ to about 1.332 g/cm³ or about 1.320 g/cm³ to about 1.332 g/cm³. In other embodiments, the densities are from about 1.050 g/cm³ to about 1.070 g/cm³, about 1.052 g/cm³ to about 1.068 g/cm³ or about 1.054 g/cm³ to about 1.066 g/cm³. In other embodiments, the densities are from about 1.080 g/cm³ to about 1.100 g/cm³, about 1.082 g/cm³ to about 1.098 g/cm³, about 1.0840 g/cm³ to about 1.096 g/cm³.

The density of liquids may be dependent on temperature. Without wanting to be bound by theory, most liquids tend to expand when temperature increases. As density is dependent on the mass and volume of the liquid, an increase in volume will cause a decrease in density. Examples of the density-temperature relationship of some solvents is given in the tables below:

TABLE 1
Density-temperature relationship of dichloromethane

| | Temperature, T (K.) | | | | | | | | | |
|---|---|---|---|---|---|---|---|---|---|---|
| | −2 | 0 | 2 | 4.5 | 6 | 8 | 10 | 13 | 15 | 17 |
| DCM | 1.362 | 1.359 | 1.355 | 1.353 | 1.349 | 1.345 | 1.343 | 1.337 | 1.334 | 1.330 |
| density | | | | | | | | | | |
| (g/ml) | | | | | | | | | | |
| | Temperature, T (K.) | | | | | | | | | |
| | 19.5 | 20 | 22 | 24.5 | 28 | 30 | 32 | 34.5 | 36.5 | 38 |
| DCM | 1.326 | 1.327 | 1.321 | 1.318 | 1.312 | 1.308 | 1.303 | 1.301 | 1.297 | 1.294 |
| density, ρ | | | | | | | | | | |
| (g/ml) | | | | | | | | | | |

TABLE 2
Density-temperature relationship of Polydimethylsiloxane fluids
(PSF-Fluids) and Diphenyl-Dimethylsiloxane fluid (DPDM-400)

| | Density, ρ (g/ml) | | | | | |
|---|---|---|---|---|---|---|
| | Temperature, T (° C.) | | | | | |
| Silicone oil | −40 | −20 | 0 | 25 | 50 | 100 |
| PSF-50 cSt | 1.020 | 1.002 | 0.982 | 0.960 | 0.938 | 0.897 |
| PSF-100 cSt | 1.024 | 1.006 | 0.987 | 0.965 | 0.944 | 0.902 |
| PSF-1,000 cSt | 1.024 | 1.006 | 0.987 | 0.965 | 0.944 | 0.902 |
| DPDM-400 | 1.029 | 1.001 | 0.992 | 0.970 | 0.949 | 0.907 |

TABLE 3
Density-temperature relationship of aqueous NaCl solutions with different concentrations

| NaCl | Density, ρ (g/ml) | | | | | | | | |
|---|---|---|---|---|---|---|---|---|---|
| Concentration | Temperature, T (° C.) | | | | | | | | |
| (wt.-%) | 0 | 10 | 20 | 25 | 30 | 40 | 50 | 60 | 70 |
| 1 | 1.007 | 1.007 | 1.005 | 1.004 | 1.002 | 0.999 | 0.995 | 0.990 | 0.979 |
| 2 | 1.015 | 1.014 | 1.012 | 1.011 | 1.010 | 1.006 | 1.001 | 0.997 | 0.972 |
| 4 | 1.030 | 1.029 | 1.026 | 1.025 | 1.024 | 1.020 | 1.015 | 1.010 | 0.984 |
| 6 | 1.046 | 1.044 | 1.041 | 1.040 | 1.038 | 1.034 | 1.029 | 1.024 | 0.999 |
| 8 | 1.061 | 1.059 | 1.055 | 1.054 | 1.052 | 1.048 | 1.043 | 1.038 | 1.013 |
| 10 | 1.077 | 1.074 | 1.071 | 1.069 | 1.067 | 1.062 | 1.058 | 1.052 | 1.028 |
| 12 | 1.092 | 1.089 | 1.086 | 1.084 | 1.082 | 1.077 | 1.072 | 1.067 | 1.042 |
| 14 | 1.108 | 1.105 | 1.101 | 1.099 | 1.097 | 1.092 | 1.087 | 1.081 | 1.057 |
| 16 | 1.124 | 1.121 | 1.116 | 1.114 | 1.112 | 1.107 | 1.102 | 1.096 | 1.071 |
| 18 | 1.140 | 1.136 | 1.131 | 1.130 | 1.127 | 1.122 | 1.117 | 1.111 | 1.086 |
| 20 | 1.157 | 1.153 | 1.148 | 1.145 | 1.143 | 1.138 | 1.132 | 1.127 | 1.102 |
| 22 | 1.173 | 1.169 | 1.164 | 1.161 | 1.159 | 1.154 | 1.148 | 1.143 | 1.117 |
| 24 | 1.190 | 1.186 | 1.180 | 1.178 | 1.175 | 1.170 | 1.164 | 1.158 | 1.133 |
| 26 | 1.207 | 1.203 | 1.197 | 1.194 | 1.192 | 1.186 | 1.180 | 1.175 | 1.149 |

TABLE 4
Density-temperature relationship of aqueous
Potassium Carbonate (K₂CO₃) solutions
with different concentrations

| K₂CO₃ | Density, ρ (g/ml) | | | | | | |
|---|---|---|---|---|---|---|---|
| Concentration | Temperature, T (° C.) | | | | | | |
| (wt.-%) | 0 | 10 | 20 | 40 | 60 | 80 | 100 |
| 1 | 1.009 | 1.009 | 1.007 | 1.001 | 0.992 | 0.980 | 0.967 |
| 2 | 1.019 | 1.018 | 1.016 | 1.010 | 1.001 | 0.989 | 0.976 |
| 4 | 1.038 | 1.037 | 1.035 | 1.028 | 1.018 | 1.006 | 0.995 |
| 8 | 1.077 | 1.075 | 1.072 | 1.064 | 1.054 | 1.042 | 1.029 |
| 12 | 1.116 | 1.113 | 1.110 | 1.101 | 1.091 | 1.079 | 1.066 |
| 16 | 1.156 | 1.153 | 1.149 | 1.140 | 1.129 | 1.117 | 1.105 |
| 20 | 1.198 | 1.194 | 1.190 | 1.181 | 1.169 | 1.157 | 1.145 |
| 24 | 1.241 | 1.237 | 1.232 | 1.222 | 1.211 | 1.199 | 1.187 |
| 28 | 1.285 | 1.280 | 1.276 | 1.265 | 1.254 | 1.242 | 1.230 |
| 30 | 1.307 | 1.303 | 1.298 | 1.287 | 1.276 | 1.264 | 1.252 |
| 35 | 1.365 | 1.360 | 1.355 | 1.344 | 1.332 | 1.321 | 1.309 |
| 40 | 1.424 | 1.420 | 1.414 | 1.403 | 1.391 | 1.380 | 1.368 |
| 45 | 1.487 | 1.482 | 1.476 | 1.464 | 1.453 | 1.441 | 1.429 |
| 50 | 1.552 | 1.546 | 1.540 | 1.529 | 1.517 | 1.505 | 1.493 |

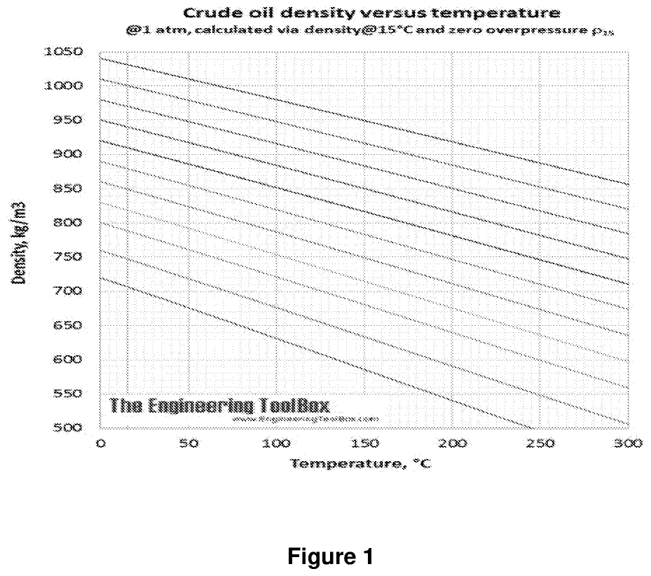

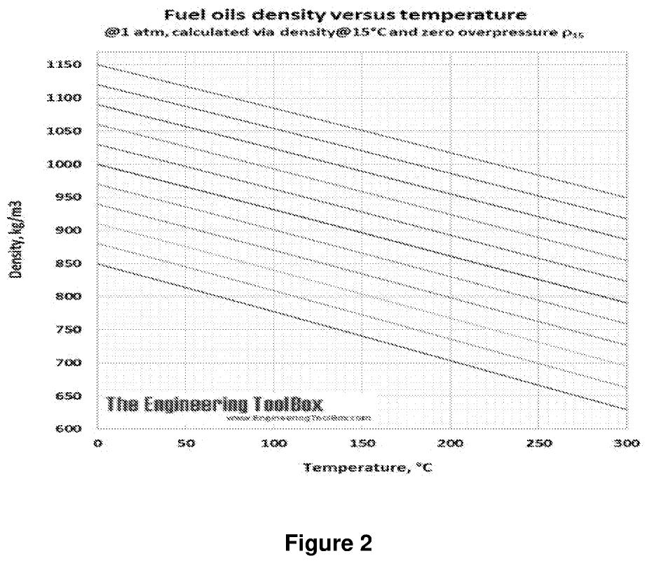

FIG. 1 further illustrates the density-temperature relationship of crude oils and FIG. 2 illustrates the density-temperature relationship of fuel oils. As the temperature changes, the density of the oils changes along the lines.

Advantageously, the emulsions formed from the density matching of the water phase and oil phase are stable. In this regard, the emulsions are kinetically stable such that no constant agitation is needed to maintain the emulsified state and the stability can be maintained for use with a shelf-life longevity of at least 5 days. In an embodiment, the emulsions are stable for at least 5 days. In this regard, the emulsion can maintain a homogenous (or emulsified) state for at least 5 days, that is, without substantially coalescencing. In an embodiment, the emulsion does not phase separate for at least 5 days. The phase separation and/or coalescence can be determined visually, or measured, for example, using light scattering techniques such as dynamic light scattering or by monitoring the absorbance or turbidity at strategic points of the emulsion. For example, the emulsion may be deemed to be phase separating and/or coalescencing when a measurement taken immediately (or within 2 hr) after forming the emulsion and a measurement taken at any later time period differs by more than about 40%. In this regard, the emulsion is considered to be stable when the difference is less than about 40%. In other embodiments, the difference is less than about 38%, about 36%, about 34%, about 32%, about 30%, about 28%, about 26%, about 24%, about 22%, about 20%, about 18%, about 16%, about 14%, about 12%, about 10%, about 8% or about 6%. In other embodiments, the emulsion is at least kinetically stable for at least 5 days. In other embodiments, the emulsion is stable for at least 6 days, at least 7 days, at least 8 days, at least 9 days, at least 10 days, at least 12 days, at least 14 days, at least 16 days, at least 18 days, at least 20 days, at least 25 days, at least 30 days, at least 35 days or at least 40 days.

The water phase may comprise other additional agents. For example, the aqueous solution may further comprise an alkali, an acid, a sugar, a cyclodextrin, a starch or a mixture thereof. The skilled person would understand, from a reading of the disclosed invention and the examples, that these agents can be added, but ultimately the density of the resultant water phase must be substantially similar (as mentioned above) the density of the oil phase.

Accordingly, in some embodiments, the emulsion comprises:
a) a water phase, the water phase is an aqueous solution comprising a salt and an additional agent; and
b) an oil phase, the oil phase comprising a polymer;

wherein the oil phase is immiscible with the water phase, and

wherein the density differential of the water phase and oil phase is less than about 0.02 g/cm³.

In some embodiments, the emulsion comprises:
a) a water phase, the water phase is an aqueous solution comprising a salt, an alkali, an acid, a sugar, a cyclodextrin, a starch or a mixture thereof; and
b) an oil phase, the oil phase comprising a polymer;

wherein the oil phase is immiscible with the water phase, and

wherein the density differential of the water phase and oil phase is less than about 0.02 g/cm³.

The aqueous salt solution may comprise one or more types of salts. These salts can be, for example, Potassium Carbonate (K₂CO₃), Potassium Acetate (K(CH₃CO₂)), Potassium Bromide (KBr), Potassium Chloride (KCl), Potassium Iodide (KI), Potassium Bisulfate (KHSO₄), Potassium Sulfate (K₂SO₄), Potassium Phosphate Monobasic (KH₂PO₄), Calcium Chloride (CaCl₂), Calcium Acetate (Ca(C₂H₃O₂)₂), Zinc Chloride (ZnCl₂), Zinc Sulfate (ZnSO₄), Sodium Acetate (Na(CH₃CO₂)), Sodium Carbonate (Na₂CO₃), Sodium Bromide (NaBr), Sodium Chloride (NaCl), Sodium Iodide (NaCI), Sodium Bisulfate (Na₂SO₄), Sodium Sulfate (Na₂SO₄), Sodium thiosulfate (sodium hyposulfite) (Na₂S₂O₃), Sodium Sulfite (Na₂SO₃), Sodium Phosphate Tribasic (Na₃PO₄), Sodium Phosphate Dibasic (Na₂HPO₄), Sodium Perchlorate (NaClO₄), Ammonium Acetate (CH₃COONH₄), Ammonium Carbonate ((NH₄)₂CO₃), Ammonium Sulfate ((NH₄)₂SO₄), Lithium Chloride (LiCl), Magnesium Chloride (MgCl₂), Magnesium Sulfate (MgSO₄), Silver Nitrate (AgNO₃), Cupric Sulfate (CuSO₄), Cesium Sulfate (Cs₂SO₄), Cesium Chloride (CsCl), and Cobaltous Chloride (CoCl₂).

In certain embodiments, the salt is selected from Potassium Carbonate (K₂CO₃) or Sodium Chloride (NaCl). In another embodiment, the salt is selected from NaCl₂, KCl, CaCl₂, MgCl₂, MgSO₄, NaHCO₃, K₂HPO₄, Na₂HPO₄, NaH₂PO₄ or Fe(NO₃)₃.

The alkali may be, for example, Sodium Hydroxide (NaOH), Potassium Hydroxide (KOH), etc. The water phase may comprise an acid solution, comprising one or more types of acids. These acids may be, for example, sulfuric acid (H₂O₄), hydrochloric acid (HCl), Acetic acid (CH₃COOH), etc. In an embodiment, the acid is selected from HCl, NaHCO₃, H₂CO₃, K₂HPO₄, KH₂PO₄, Na₂HPO₄ or NaH₂PO₄.

The sugar may be Fructose, Glucose, or Sucrose.

In an embodiment, the water phase is a solution having a salt or sucrose concentration up to about 1000 g/L. In other embodiments, the water phase is an aqueous solution having a salt concentration from about 10 g/L to about 1000 g/L. In other embodiments, the concentration is from about 10 g/L to about 950 g/L. In other embodiments, the concentration is from about 10 g/L to about 900 g/L. In other embodiments, the concentration is from about 10 g/L to about 850 g/L. In other embodiments, the concentration is from about 10 g/L to about 800 g/L. In other embodiments, the concentration is about 10 g/L, about 50 g/L, about 100 g/L, about 110 g/L, about 120 g/L, about 130 g/L, about 140 g/L, about 150 g/L, about 160 g/L, about 170 g/L, about 180 g/L, about 190 g/L, about 200 g/L, about 210 g/L, about 220 g/L, about 230 g/L, about 240 g/L, about 250 g/L, about 260 g/L, about 270 g/L, about 280 g/L, about 290 g/L, about 300 g/L, about 310 g/L, about 320 g/L, about 330 g/L, about 340 g/L, about 350 g/L, about 360 g/L, about 370 g/L, about 380 g/L, about 390 g/L, about 400 g/L, about 410 g/L, about 420 g/L, about 430 g/L, about 440 g/L, about 450 g/L, about 460 g/L, about 470 g/L, about 480 g/L, about 490 g/L, about 500 g/L, about 510 g/L, about 520 g/L, about 530 g/L, about 540 g/L, about 550 g/L, about 560 g/L, about 570 g/L, about 580 g/L, about 590 g/L about 600 g/L, about 620 g/L, about 640 g/L, about 660 g/L, about 680 g/L, about 700 g/L, about 720 g/L, about 740 g/L, about 760 g/L, about 780 g/L, about 800 g/L, about 820 g/L, about 840 g/L, about 860 g/L, about 880 g/L, about 900 g/L, about 920 g/L, about 940 g/L, about 960 g/L, about 980 g/L or about 1000 g/L. In an embodiment, the water phase has a density from about 1 g/cm³ to about 1.7 g/cm³. In other embodiments, the density is from about 1.004 g/cm³ to about 1.68 g/cm³. In other embodiments, the density is from about 1.004 g/cm³ to about 1.66 g/cm³. In other embodiments, the density is from about 1.004 g/cm³ to about 1.64 g/cm³. In other embodiments, the density is from about 1.004 g/cm³ to about 1.62 g/cm³. In other embodiments, the density is from about 1.004 g/cm³ to about 1.6 g/cm³. In other embodiments, the density is from about 1.004 g/cm³ to about 1.58 g/cm³. In other embodiments, the density is from about 1.004 g/cm³ to about 1.56 g/cm³. In other embodiments, the density is from about 1.004 g/cm³ to about 1.54 g/cm³. In other embodiments, the density is from about 1.004 g/cm³ to about 1.52 g/cm³. In other embodiments, the density is from about 1.004 g/cm³ to about 1.5 g/cm³.

In an embodiment, the water phase is free of emulsifier. In another embodiment, the water phase is free of surfactant. Surfactants are entities that lower tension between a surface and a liquid or between two or more immiscible substances. Surfactants operate by segregating at the interface between oil and water phases. They do this by having molecules with an oil-loving tail and a water-loving head. Emulsifier is a subset of surfactant, and refers to an entity that stabilizes an emulsion.

The oil phase comprises a polymer. The polymer can be selected from polycaprolactone (PCL) and its copolymer, polylactic acid (PLA) and its copolymer, polyglycolide (PGA) and its copolymer such as poly(lactic-co-glycolic acid) (PLGA), poly(glycolide-co-caprolactone), poly (glycolide-co-trimethylene carbonate), polystyrene (PS), or polyethylene terephthalate (PET). The polymer can be a block copolymer of the above mentioned polymers. The polymer can be mixture or blend of the above mentioned polymers. The skilled person would understand that other suitable polymers, including amphiphilic block copolymers may be used.

In certain embodiments, the polymer is selected from polycaprolactone (PCL) and its copolymer.

As mentioned above, the oil phase refers to an organic based solvent or solvent system, and which comprises of mainly organic solvent. In this regard, the polymer is dissolvable or soluble in an organic based solvent or solvent system, which can be an oil, organic solvent or mixture thereof. In this regard, the oil phase can have a density of about >1.0 g/cm³. Corollary, the polymer is insoluble in the water phase.

In an embodiment, the organic solvent is selected from dichloromethane (DCM), chloroform, 1,1-Dichloroethane, 1,2-dichloroethane, brominated vegetable oil (BYO), ester gum (EG), damar gum (DG), sucrose acetate isobutyrate (SAIB), animal oil, vegetable oil, mineral oil, silicone oil or a mixture thereof. In certain embodiments, the organic solvent is DCM.

In another embodiment, the density of the oil phase is from about 1 g/cm³ to about 1.7 g/cm³. In other embodiments, the density is from about 1 g/cm³ to about 1.68 g/cm³. In other embodiments, the density is from about 1 g/cm³ to about 1.66 g/cm³. In other embodiments, the density is from about 1 g/cm³ to about 1.64 g/cm³. In other embodiments, the density is from about 1 g/cm³ to about 1.62 g/cm³. In other embodiments, the density is from about 1 g/cm³ to about 1.6 g/cm³. In other embodiments, the density is from about 1 g/cm³ to about 1.58 g/cm³. In other embodiments, the density is from about 1 g/cm³ to about 1.56 g/cm³. In other embodiments, the density is from about 1 g/cm³ to about 1.54 g/cm³. In other embodiments, the density is from about 1 g/cm³ to about 1.52 g/cm³. In other embodiments, the density is from about 1 g/cm³ to about 1.5 g/cm³.

In some embodiments, the polymer has a concentration from about 10 mg/mL to about 400 mg/mL. In other embodiments, the concentration is from about 10 mg/mL to about 350 mg/mL, from about 10 mg/mL to about 300 mg/mL, from about 10 mg/mL to about 250 mg/mL, from about 15 mg/mL to about 250 mg/mL, from about 20 mg/mL to about 250 mg/mL, from about 30 mg/mL to about 250 mg/mL, from about 40 mg/mL to about 250 mg/mL, from about 50 mg/mL to about 250 mg/mL, from about 60 mg/mL to about 250 mg/mL, from about 70 mg/mL to about 250 mg/mL, from about 80 mg/mL to about 250 mg/mL, from about 90 mg/mL to about 250 mg/mL, or from about 100 mg/mL to about 250 mg/mL.

In an embodiment, the oil phase is free of emulsifier. In another embodiment, the oil phase is free of surfactant.

In some embodiments, the emulsion does not have an emulsifier. In this regard, the emulsion is free of emulsifier. In other embodiments, the emulsion does not have a surfactant. In this regard, the emulsion is free of surfactant. The skilled person would know that, based on the teachings of the present invention, emulsifiers and surfactants are not required for the emulsion to perform its desired function. However, emulsifiers and surfactant may optionally be added to further improve the stability of the emulsion. Accordingly, in an embodiment, the emulsion further comprises an emulsifier.

In some embodiments, the average size of the dispersed phase (or particles) in the emulsion is of about 0.8 μm to about 3 μm. In other embodiments, the average size is about 0.9 μm to about 3 μm, about 1 μm to about 3 μm, about 1 μm to about 2.8 μm, about 1 μm to about 2.6 μm, about 1 μm to about 2.4 μm, about 1 μm to about 2.2 μm, about 1 μm to about 2 μm, about 1 μm to about 1.8 μm, about 1 μm to about 1.6 μm or about 1 μm to about 1.5 μm. In other embodiments, the average size is about 0.1 μm, about 0.2 μm, about 0.3 μm, about 0.4 μm, about 0.5 μm, about 0.6 μm, about 0.7 μm, about 0.8 μm, about 0.9 μm, about 1 μm, about 1.1 μm, about 1.2 μm, about 1.3 μm, about 1.4 μm, about 1.5 μm, about 1.6 μm, about 1.7 μm, about 1.8 μm, about 1.9 μm, about 2 μm, about 2.2 μm, about 2.4 μm, about 2.6 μm, about 2.8 μm, about 3 μm, about 4 μm, about 5 μm, about 6 μm, about 7 μm, about 8 μm, about 9 μm.

The skilled person would understand that the teachings of the present invention can be applied to either a w/o emulsion or a o/w emulsion.

In some embodiments, the emulsion comprises:
a) a water phase at less than 50 vol % of the final emulsion volume; and
b) an oil phase at more than 50 vol % of the final emulsion volume.

In this regard, the emulsion is a w/o emulsion. In an embodiment, the water phase is less than 50% of the final emulsion volume, less than about 45%, less than about 40%, less than about 35%, less than about 30%, less than about 25%, less than about 20%, less than about 15%, less than about 10% or less than about 5%.

In another embodiment, the oil phase is more than 50% of the final emulsion volume, more than about 55%, more than about 60%, more than about 65%, more than about 70%, more than about 75%, more than about 80%, more than about 85%, more than about 90% or more than about 95%.

The inventors have further found that a w/o emulsion can be formed when the volume fraction of the water phase is less than 70%. Accordingly, in another embodiment, the emulsion is a w/o emulsion comprising a water phase at less than 70 vol % of the final emulsion volume and an oil phase at more than 30 vol % of the final emulsion volume. In an embodiment, the water phase is less than 70% of the final emulsion volume, less than about 65%, less than about 60%, less than about 55% or less than about 50%. In another embodiment, the oil phase is more than 30% of the final emulsion volume, more than about 35%, more than about 40%, more than about 45% or more than about 50%.

In an embodiment, the emulsion comprises:
a) a water phase at more than 50 vol % of the final emulsion volume; and
b) an oil phase at less than 50 vol % of the final emulsion volume.

In this regard, the emulsion is a o/w emulsion. In an embodiment, the oil phase is less than 50% of the final emulsion volume, less than about 45%, less than about 40%, less than about 35%, less than about 30%, less than about 25%, less than about 20%, less than about 15%, less than about 10% or less than about 5%.

In another embodiment, the water phase is more than 50% of the final emulsion volume, more than about 55%, more than about 60%, more than about 65%, more than about 70%, more than about 75%, more than about 80%, more than about 85%, more than about 90% or more than about 95%.

The inventors have further found that a o/w emulsion can be formed when the volume fraction of the oil phase is less than 70%. Accordingly, in another embodiment, the emulsion is a o/w emulsion comprising a oil phase at less than 70 vol % of the final emulsion volume and an water phase at more than 30 vol % of the final emulsion volume. In an embodiment, the oil phase is less than 70% of the final emulsion volume, less than about 65%, less than about 60%, less than about 55% or less than about 50%. In another embodiment, the water phase is more than 30% of the final emulsion volume, more than about 35%, more than about 40%, more than about 45% or more than about 50%.

In an embodiment, the emulsion comprises:
a) a water phase, the water phase is an aqueous solution comprising a salt; and
b) an oil phase, the oil phase comprising a polymer;

wherein the oil phase is immiscible with the water phase,

wherein the polymer is polycaprolactone (PCL) or its copolymer, and

wherein the density differential of the water phase and oil phase is less than about 0.02 g/cm³.

The emulsion can further comprise an outer continuous phase to form a double emulsion. In some embodiments, the outer continuous phase comprises a dispersant from about 0.1 wt % to about 20 wt % of the outer continuous phase and an emulsifier from about 0.1 wt % to about 20 wt % of the outer continuous phase. In this regard, a w/o emulsion can further comprising an outer aqueous/water continuous phase (w) to form a w/o/w double emulsion. The outer water phase (w₂) can be an aqueous solution comprising dispersants or/and emulsifiers. For example, dispersants such as Polyvinyl alcohol (PVA), or non-ionic emulsifiers with HLB value >6, for example, Pluronic® F-127, Tween 80 can be used. The outer water phase can comprise a dispersant or non-ionic emulsifier with a concentration from zero to about 10.0 wt % of the outer water phase. In another embodiment, concentration is less than about 20 wt %, about 18 wt %, about 16 wt %, about 14 wt %, about 12 wt %, about 10 wt %, about 9 wt %, about 8 wt %, about 7 wt %, about 6 wt %, about 5 wt %, about 4 wt % or about 3 wt %. In this regard, PVA, F-127 or Tween 80 independently have a concentration from zero to about 20 wt %.

Other known emulsifiers can be used, such as Polyoxyethylene (20) sorbitan monooleate (Polysorbate 80, Tween 80) or Polysorbate 60. Emulsifier with a HLB value >6 that can be used, are Ethoxylated aliphatical alcohol, polyoxyethylene surfactants, carboxylic esters, polyethylene glycol esters, anhydrosorbitol ester and its ethoxylated derivatives, glycol esters of fatty acids, carboxylic amides, monoalkanolamine condensates, polyoxyethylene fatty acid amides, for examples, Diethylene glycol monolaurate, Diethylene glycol fatty acid ester, Sorbitan monopahnitate, Polyoxyethylene dioleate, Tetraethylene glycol monooleate, Polyoxypropylene annitol dioleate, Polyoxyethylene sorbitol lanolin oleate derivative, Polyoxyethylene sorbitol lanolin derivative, Polyoxypropylene stearate, Sorbitan monolaurate, Polyoxyethylene fatty acid, Polyoxyethylene sorbitol beeswax derivative, Polyoxyethylene oxypropylene oleate, Tetraethylene glycol monolaurate, Polyoxyethylene lauryl ether, Polyoxyethylene sorbitan monostearate, Hexaethylene glycol monostearate, Polyoxyethylene sorbitan monooleate, Polyoxyethylene cetyl ether, Polyoxyethylene sorbitan tristearate, Polyoxyethylene sorbitan trioleate, Polyoxyethylene monooleate, Polyoxyethylene monostearate, Polyoxyethylene monopalmitate, Polyoxyethylene monolaurate, Polyoxyethylene alkyl aryl ether, Polyoxyethylene vegetable oil, Polyoxyethylene sorbitan monolaurate, Polyoxyethylene sorbitan monopalmitate, Polyoxyethylene cetyl alcohol, poly(ethylene oxide)-poly(propylene oxide)-poly(ethylene oxide)(PEO-PPO-PEO), fatty alcohol polyethylene oxide ether, linear alcohol polyethylene oxide ether, sorbitan monolaurate, nonyl phenyl polyethylene oxide ether, sorbitan monolaurate polyethylene oxide ether, or mixture thereof.

Surfactants with HLB value <6 that can be used, for examples but not limited to, are Sorbitan trioleate, Polyoxyethylene sorbitol beeswax derivative, Sorbitan tristearate, Polyoxyethylene sorbitol hexastearate, Ethylene glycol fatty acid ester, Propylene glycol fatty acid ester, Propylene glycol monostearate, Propylene glycol monostearate, Sorbitan sesquioleate, Glycerol monosrearate, Sorbitan monooleate, Propylene glycol monolaurate, Diethylene glycol monooleate, Diethylene glycol fatty acid ester, Diethylene glycol monostearate, Diethylene glycol fatty acid ester, Diethylene glycol fatty acid ester, Glycerol monostearate, Diethylene glycol fatty acid ester), for examples, mixtures of sorbitan monoester (Span) and polyethoxylated sorbitan monoester (such as Polyoxyethylene (20) sorbitan monooleate (Polysorbate 80, Tween 80)), or Polysorbate 60.

In an embodiment, the o/w emulsion further comprises an outer oil continuous phase (o) to form a o/w/o double emulsion. Surfactants with HLB value <6, as mentioned above, can be used in the outer continuous oil phase.

The density-matched emulsion can be a product per-se or used an intermediate as a material and/or within across step in the development/fabrication of other products. The emulsion of the present disclosure can be used in paints and pigments. The stable emulsions of the present invention can be used in various applications, such as generating high porosity polymer particles, for use as templates for porous materials or for drug encapsulation.

The emulsion as disclosed herein can also be used in cosmetics. For example, the emulsions can be used for delivering specific molecules, pigment particles or active ingredient to the targeted site. The emulsions can be used in different types of cosmetics and include products intended to cleanse or beautify. As such, cosmetics refer to products for use in beauty and personal care. As used herein, cosmetics is used in the broadest sense and can include, but not limited to, sunscreen, lipstick, lip gloss, lip liner, lip plumper, lip balm, lip stain, lip conditioner, lip primer, lip booster, lip butters, foundations, primers, concealer, blush powder, bronzer, setting spray, contour cream/powder, mascara, mascara primer, eye shadow, eye primer, eyelash glue, eyelash curler, eyebrow pencils, creams, waxes, gels, nail polish, nail gloss, and cleansing products such as makeup remover, toners, facial masks, exfoliants, moisturizers, soap, hand wash or shampoo.

In an aspect, the present invention provides a method of making an emulsion, comprising mixing a water phase and an oil phase with substantially similar densities. In this regard, the water phase and the oil phase have densities as disclosed above.

In some embodiments, the method of making an emulsion comprises the steps of:
a) homogenising a water phase and an oil phase to form an emulsion, the water phase is an aqueous solution comprising a salt, the oil phase comprising a polymer;

wherein the oil phase is immiscible with the water phase, and

wherein the density differential of the water phase and oil phase is less than about 0.02 g/cm³.

In other embodiments, the method of making an emulsion comprises the steps of:
a) homogenising a water phase and an oil phase to form an emulsion, the water phase is an aqueous solution comprising a salt, an alkali, an acid, a sugar, a cyclodextrin, a starch or a mixture thereof, and the oil phase comprising a polymer;

wherein the oil phase is immiscible with the water phase, and

wherein the density differential of the water phase and oil phase is less than about 0.02 g/cm³.

In this method, the water phase and the oil phase are homogenised. In this regard, the two phases are mixed such that the two mutally non-miscible liquids are the same throughout. This is achieved by turning one of the phase into a state consisting of small particles distributed uniformly throughout the other phase. The skilled person would know that the phases can be homogenised using any technique known in the art. For example, homongenisation can be performed using a homogeniser, ultrasonic homogeniser, dispersers, stirrers, or ultrasound. The homogenisation process can be further aided by adding one phase into another during the process. For example, to generate a w/o emulsion, the water phase (aqueous salt solution) can be added into the oil phase (polymer solution) during the homogenisation process.

Microfluidic devices can subsequently be used to generate w₁/o/w₂ droplets (double emulsion), which are then collected and solidified to be porous polymer microspheres in an aqueous solution with ethanol, dispersants or/and emulsifying additives. As examples, the porous polymer microspheres described below employed the polymer of polycaprolactone (PCL), sourced from Sigma-Aldrich (Ref. 704105), with average Mₙ 45,000. These porous PCL spheres have a diameter of 1-500 micrometers, a high porosity in the range of 10-90%, and a density below 1.1 g/cm³, achieved by using 10-250 mg/ml PCL solution to prepare the spheres. Other grades of PCL, including medical grade PCL, potentially composed of different molecular weight distributions, may similarly be used to fabricate microspheres, as described herein.

In an embodiment, the method further comprises flowing the emulsion into the outer water or oil continuous phase in a fluidic system, for forming a double emulsion. This is, for example, exemplified in FIG. 4, wherein a w/o emulsion is flowed into a water continuous phase to form a w/o/w emulsion.

In some embodiments, the method further comprises the step of flowing the emulsion into an outer continuous phase to form a double emulsion, the outer continuous phase comprising a dispersant from about 0.1 wt % to about 10 wt % of the outer continuous phase and an emulsifier from about 0.1 wt % to about 10 wt % of the outer continuous phase.

In an embodiment, the dispersant concentration is from 0.1 wt % to about 20 wt %. In another embodiment, the dispersant is less than about 20 wt % of the outer continuous phase, less than about 18 wt %, less than about 16 wt %, less than about 14 wt %, less than about 12 wt %, less than about 10 wt %, less than about 9 wt %, about 8 wt %, about 7 wt %, about 6 wt %, about 5 wt %, about 4 wt %, about 3 wt % or about 2 wt %. In other embodiments, the dispersant is about 0.1 wt % to about 20 wt % of the solvent exchange liquid, about 0.1 wt % to about 18 wt %, about 0.1 wt % to about 16 wt %, about 0.1 wt % to about 14 wt %, about 0.1 wt % to about 12 wt %, about 0.1 wt % to about 10 wt %, about 0.1 wt % to about 9 wt %, about 0.1 wt % to about 8 wt %, about 0.1 wt % to about 7 wt %, about 0.1 wt % to about 6 wt %, about 0.1 wt % to about 5 wt %, about 0.1 wt % to about 4 wt %, about 0.1 wt % to about 3 wt %, about 0.1 wt % to about 2 wt %, about 0.5 wt % to about 2 wt % or about 1 wt % to about 2 wt %.

In an embodiment, the emulsifier concentration is from about 0.1 wt % to about 20 wt %. In another embodiment, the emulsifier is less than about 20 wt % of the outer continuous phase, less than about 18 wt %, less than about 16 wt %, less than about 14 wt %, less than about 12 wt %, less than about 10 wt %, less than about 9 wt %, about 8 wt %, about 7 wt %, about 6 wt %, about 5 wt %, about 4 wt %, about 3 wt % or about 2 wt %. In other embodiments, the emulsifier is about 0.1 wt % to about 20 wt % of the solvent exchange liquid, about 0.1 wt % to about 18 wt %, about 0.1 wt % to about 16 wt %, about 0.1 wt % to about 14 wt %, about 0.1 wt % to about 12 wt %, about 0.1 wt % to about 10 wt %, about 0.1 wt % to about 9 wt %, about 0.1 wt % to about 8 wt %, about 0.1 wt % to about 7 wt %, about 0.1 wt % to about 6 wt %, about 0.1 wt % to about 5 wt %, about 0.1 wt % to about 4 wt %, about 0.1 wt % to about 3 wt %, about 0.5 wt % to about 3 wt %, about 1 wt % to about 3 wt % or about 2 wt % to about 3 wt %.

In another embodiment, the method further comprises immersing the w/o/w or o/w/o emulsion (double emulsion) in a solvent exchange liquid to form polymer microspheres. The polymer are provided in the w/o or o/w emulsion, and can be in the water or oil phase. As illustrated in FIG. 4, by allowing the emulsion to flow into an immiscible outer continuous phase, the emulsion is maintained in a globular state and when transferred into a solvent exchange liquid, this globular state is maintained. Without wanting to be bound by theory, the solvent exchange liquid works through a process of liquid-liquid extraction, which is based on the relative solubilities of immiscible liquids to separate compounds or entities. This results in a net transfer of one or more species from one liquid into another liquid phase. By applying this concept to the solvent exchange liquid, the solvents in the globule of the emulsion can be extracted out. Additionally, other entities such as the salt, sugar, acid, dispersant and/or emulsifiers can also be extracted from the globule of the emulsion. Accordingly, the polymer in the emulsion can undergo surface aggregation as it is exposed to a non-solvent. In this diffusion process (i.e. occurs from the surface of the globule and slowly penetrates the core), the globular state of the emulsion is maintained and transformed into a spherical form, such that the resultant polymer (which is not miscible with the solvents) is retained as a microsphere.

In an embodiment, the solvent exchange liquid comprises dispersants, non-ionic emulsifiers, alcohol, and acid. The dispersants can be polyvinyl alcohol (PVA). Non-ionic emulsifiers with HLB value >6 may be used, for example: Pluronic F-127, or Tween 80. Examples of alcohol can be ethanol, methanol, or isopropyl alcohol (IPA). Acid is for example hydrochloric acid (HCl), or sulfuric acid (H₂SO₄). The dispersant, emulsifiers, alcohol and acid act to facilitate the diffusion of solvent from the double emulsion out into the solvent exchange liquid.

In an embodiment, the dispersant concentration is from zero to about 20 wt %. In another embodiment, the dispersant is less than about 20 wt % of the solvent exchange liquid, less than about 18 wt %, less than about 16 wt %, less than about 14 wt %, less than about 12 wt %, less than about 10 wt %, less than about 9 wt %, about 8 wt %, about 7 wt %, about 6 wt %, about 5 wt %, about 4 wt %, about 3 wt % or about 2 wt %. In other embodiments, the dispersant is about 0.1 wt % to about 20 wt % of the solvent exchange liquid, about 0.1 wt % to about 18 wt %, about 0.1 wt % to about 16 wt %, about 0.1 wt % to about 14 wt %, about 0.1 wt % to about 12 wt %, about 0.1 wt % to about 10 wt %, about 0.1 wt % to about 9 wt %, about 0.1 wt % to about 8 wt %, about 0.1 wt % to about 7 wt %, about 0.1 wt % to about 6 wt %, about 0.1 wt % to about 5 wt %, about 0.1 wt % to about 4 wt %, about 0.1 wt % to about 3 wt %, about 0.1 wt % to about 2 wt %, about 0.5 wt % to about 2 wt % or about 1 wt % to about 2 wt %.

In an embodiment, the emulsifier concentration is from zero to about 20 wt %. In another embodiment, the emulsifier is less than about 20 wt % of the solvent exchange liquid, less than about 18 wt %, less than about 16 wt %, less than about 14 wt %, less than about 12 wt %, less than about 10 wt %, less than about 9 wt %, about 8 wt %, about 7 wt %, about 6 wt %, about 5 wt %, about 4 wt %, about 3 wt % or about 2 wt %. In other embodiments, the emulsifier is about 0.1 wt % to about 20 wt % of the solvent exchange liquid, about 0.1 wt % to about 18 wt %, about 0.1 wt % to about 16 wt %, about 0.1 wt % to about 14 wt %, about 0.1 wt % to about 12 wt %, about 0.1 wt % to about 10 wt %, about 0.1 wt % to about 9 wt %, about 0.1 wt % to about 8 wt %, about 0.1 wt % to about 7 wt %, about 0.1 wt % to about 6 wt %, about 0.1 wt % to about 5 wt %, about 0.1 wt % to about 4 wt %, about 0.1 wt % to about 3 wt %, about 0.5 wt % to about 3 wt %, about 1 wt % to about 3 wt % or about 2 wt % to about 3 wt %.

In an embodiment, the alcohol concentration is from zero to about 60 wt %. In another embodiment, the alcohol is less than about 80 wt % of the solvent exchange liquid, less than about 75 wt %, less than about 70 wt %, less than about 65 wt %, less than about 60 wt %, less than about 55 wt %, less than about 50 wt %, less than about 45 wt %, less than about 40 wt %, less than about 35 wt %, less than about 30 wt %, less than about 25 wt % or less than about 20 wt %. In other embodiments, the alcohol is about 0.1 wt % to about 80 wt % of the solvent exchange liquid, about 0.1 wt % to about 75 wt %, about 0.1 wt % to about 70 wt %, about 0.1 wt % to about 65 wt %, about 0.1 wt % to about 60 wt %, about 0.1 wt % to about 55 wt %, about 0.1 wt % to about 50 wt %, about 0.1 wt % to about 45 wt %, about 0.1 wt % to about 40 wt %, about 0.1 wt % to about 35 wt %, about 0.1 wt % to about 30 wt %, about 0.1 wt % to about 25 wt % or about 0.1 wt % to about 20 wt %.

In an embodiment, the acid concentration is from zero to about 10 mol/L. In another embodiment, the acid is less than about 10 M, less than about 9 M, less than about 8 M, less than about 7 M, less than about 6 M, less than about 5 M, less than about 4 M, less than about 3 M, less than about 2 M or less than about 10 M.

In an embodiment, the solvent exchange liquid has a volume of about 0.0005 litres to about 100,000 litres. In other embodiments, the volume is about 0.005 litres to about 100,000 litres, about 0.05 litres to about 100,000 litres, about 0.5 L to about 50,000 L, about 0.5 L to about 10,000 L, about 0.5 L to about 5,000 L, about 0.5 L to about 1,000 L, about 0.5 L to about 500 L, about 0.5 L to about 100 L, about 0.5 L to about 50 L, about 0.5 L to about 10 L, about 0.5 L to about 9 L, about 0.5 L to about 8 L, about 0.5 L to about 7 L, about 0.5 L to about 6 L, about 0.5 L to about 5 L, about 0.5 L to about 4 L, about 0.5 L to about 3 L, about 0.5 L to about 2 L or about 0.5 L to about 1 L.

In an embodiment, the solvent exchange liquid is in a container with a height of about 0.05 m to about 20 m. In other embodiments, the container has a height of about 0.1 m to about 20 m, about 0.1 m to about 18 m, about 0.1 m to about 16 m, about 0.1 m to about 14 m, about 0.1 m to about 12 m, about 0.1 m to about 10 m, about 0.1 m to about 8 m, about 0.1 m to about 6 m, about 0.1 m to about 4 m, about 0.1 m to about 2 m, about 0.1 m to about 1 m, about 0.1 m to about 0.8 m, about 0.1 m to about 0.6 m, about 0.1 m to about 0.4 m or about 0.1 m to about 0.2 m.

For example, to collect a small amount of w₁/o/w₂ droplets with small size (micrometer-sized) or/and make a small amount of microspheres, a volume of 0.0005 litres and the container height of 0.05 meters maybe be sufficient. If we want to collect a large number of w₁/o/w₂ droplets or/and make a large number of microspheres, the aqueous solution in the cylinder need a huge volume and height.

In some embodiments, the method of forming a polymeric microsphere, comprises the steps of:
a) homogenising a water phase and an oil phase to form an emulsion, the water phase is an aqueous solution comprising a salt, the oil phase comprising a polymer, wherein the oil phase is immiscible with the water phase, and wherein the density differential of the water phase and oil phase is less than about 0.02 g/cm³;
b) flowing the emulsion into an outer continuous phase to form a double emulsion, the outer continuous phase comprising a dispersant from about 0.1 wt % to about 20 wt % of the outer continuous phase and an emulsifier from about 0.1 wt % to about 20 wt % of the outer continuous phase; and
c) immersing the double emulsion in a solvent exchange liquid to form the polymeric microsphere.

In other embodiments, the method of forming a polymeric microsphere comprises the steps of:
a) homogenising a water phase and an oil phase to form an emulsion, the water phase is aqueous solution comprising a salt, an alkali, an acid, a sugar, a cyclodextrin, a starch or a mixture thereof, the oil phase comprising a polymer, wherein the oil phase is immiscible with the water phase, and wherein the density differential of the water phase and oil phase is less than about 0.02 g/cm³;
b) flowing the emulsion into an outer continuous phase to form a double emulsion, the outer continuous phase comprising a dispersant from about 0.1 wt % to about 20 wt % of the outer continuous phase and an emulsifier from about 0.1 wt % to about 20 wt % of the outer continuous phase; and
c) immersing the double emulsion in a solvent exchange liquid to form the polymeric microsphere.

In an aspect, the present invention discloses a polymeric microsphere obtained according to the method disclosed herein. The polymeric microsphere may have a porosity of about 10% to about 90%. The variance of the microsphere particle size distribution is less than about 0.8.

In an embodiment, the present invention provides a porous polymeric material made by the methods as disclosed herein.

In another aspect, the present invention discloses a polymeric microsphere. The polymeric microsphere may have a porosity of about 10% to about 90%. The variance of the microsphere particle size distribution is less than about 0.8.

The polymeric microsphere formed from this method is porous. The porosity of the microsphere depends on several factors, such as the ratio of water to oil in the the emulsion, the entities in the water and oil phase, and the amount of polymer used. The size of the microspheres can depend on numerous factors, such as the flow rate of the continuous phase, the flow rate of the emulsion into the continuous phase, the bore size of the outlet which introduces the emulsion into the continuous phase, the type of the emulsion, the homogenisation process of the emulsion. The multitude of factors makes this art less simplistically predictable as a small change in a parameter (especially upstream) can result in a significantly different end product.

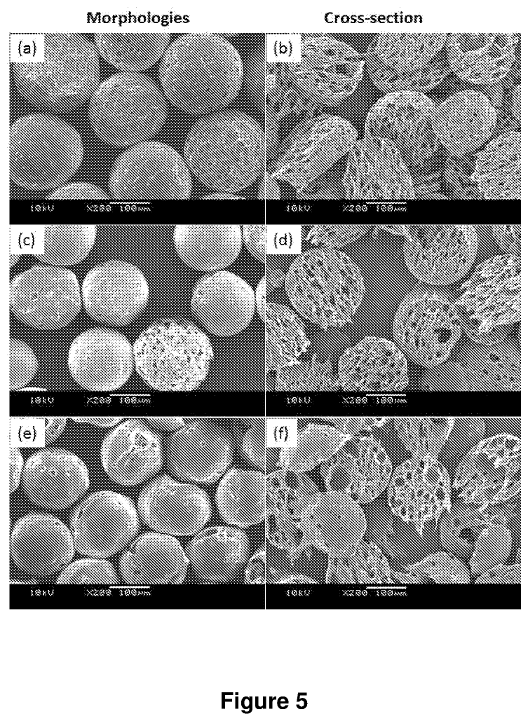

Advantagously, the inventors have found that as a result of the density matching of the emulsion (i.e. the water phase and oil phase are substantially similar), there is good consistency of the polymer microsphere morphologies throughout the production process. In this regard, the size of the microspheres, the porosity of the microspheres and the amount of polymer contained in each microsphere is consistent throughout. This is illustrated in FIG. 5, where the size of the microspheres are shown to be very homogeneous (the coefficient of variation of the microsphere are less than 10%). It is believed that the density matching allows the emulsion to be stable for a longer period of time, and as a result, flocculation, creaming, coalescening and/or demulsification does not occur (or occurs to a reduced extent) during the microsphere formation process, especially during the formation of the double emulsion. This is advantageous in a production process, where the emulsion may be required to be stored in a large tank for a long period of time. Further, production cost can be reduces as less energy input is needed to constantly agitate the storage tank so that flocculation, creaming, coalescening and/or demulsification does not occur (or occur at a reduced extent).

In this regard, the present disclosure also relates to a polymeric material made by the methods as disclosed herein. The polymeric material can be a polymeric microsphere. The polymeric material can be porous. Depending on the amount of polymer added to the emulsion and the type of emulsion, the polymeric microsphere can be a polymeric microshell or a polymeric microparticle. For example, if the emulsion is a w/o emulsion and the polymer is in the oil phase, a polymeric microshell will result (i.e. with a central cavity). If the emulsion is a o/w emulsion and the polymer is in the oil phase, a polymer microparticle will result (i.e. with a solid core). In this regard, the polymer surface aggregates to form a porous microshell with a cavity. The thickness of the microshell can accordingly be controlled by the amount of polymer used in the emulsion. In some embodiments, the thickness of the microshell is of about 0.08 μm to about 80 μm. In other embodiments, the thickness is about 0.09 μm to about 80 μm, 0.1 μm to about 80 μm, 0.1 μm to about 78 μm, 0.1 μm to about 76 μm, 0.1 μm to about 74 μm, 0.1 μm to about 72 μm, 0.1 μm to about 70 μm, 0.1 μm to about 68 μm or 0.1 μm to about 66 μm.

In an embodiment, the microsphere has a porosity of about 10% to about 90%. In this regard, porosity refers to the void fraction, and is a measure of the void spaces in a material. The skilled person would understand that it is a fraction of the volume of voids over the total volume, between 0 and 1, or as a percentage between 0% and 100%. In other embodiments, the porosity is about 15% to about 90%, about 20% to about 90%, about 30% to about 90%, about 30% to about 85%, about 30% to about 80%, about 35% to about 80%, about 40% to about 80%, about 45% to about 80%, about 50% to about 80%, about 55% to about 80%, about 60% to about 80% or about 65% to about 80%.

Because of the density matching of the emulsion, a tight distribution of polymer microsphere/microshells can be obtained. In some embodiments, the variance of the particle size distribution is less than about 0.8. The skilled person would know that the standard deviation is the square root of the variance, and defines the spread of the particles. The larger then variance, the more spread out the particle sizes are around the mean size. This is not advantageous in a production process where a tight distribution is usually desired, and can lead to increased production cost and decreased yields, when size-selection methods are applied. In other embodiments, the variance is less than about 0.7, less than about 0.6, less than about 0.5, less than about 0.4, less than about 0.3, less than about 0.2, less than about 0.1 or less than about 0.05.

Alternatively, in some embodiments, the coefficient of variation of the microspheres is less than about 40%. The skilled person would know that the coefficient of variation is also known as the relative standard deviation, which is defined as the ratio of the standard deviation to the mean. In other embodiments, the coefficient of variation is less than about 38%, about 36%, about 34%, about 32%, about 30%, about 28%, about 26%, about 24%, about 22%, about 20%, about 18%, about 16%, about 14%, about 12%, about 10%, about 8%, about 6%, about 5%.

In an embodiment, the polymer is selected from polycaprolactone (PCL) and its copolymer, polylactic acid (PLA) and its copolymer, polyglycolide (PGA) and its copolymer such as poly(lactic-co-glycolic acid) (PLGA), poly(glycolide-co-caprolactone), poly (glycolide-co-trimethylene carbonate), polystyrene (PS), or polyethylene terephthalate (PET). The polymer can be a block copolymer of the above mentioned polymers. The polymer can be mixture or blend of the above mentioned polymers. The skilled person would understand that other suitable polymers, including amphiphilic block copolymers may be used. In certain embodiments, the polymer is selected from polycaprolactone (PCL) and its copolymer. In certain embodiments, the polymer is polycaprolactone (PCL).

In some embodiments, the microsphere has an outer diameter (or particle size) of about 100 μm to about 500 μm. In other embodiments, the outer diameter is from about 120 μm to about 480 μnm, about 140 μm to about 460 μm, about 160 μm to about 440 μm, about 180 μm to about 420 μm, about 200 μm to about 400 μm, about 220 μm to about 380 μm, about 240 μm to about 360 μm or about 260 μm to about 320 μm.

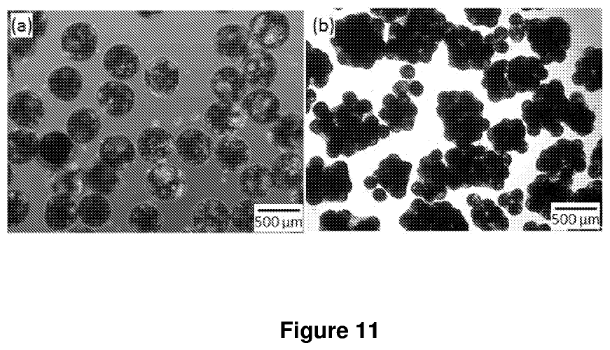

The porous polymer microspheres can be used as microcarriers for cell culture. This is exemplified in the examples and in FIG. 11-13. These emulsions and microspheres can encapsulate drugs or other bioactive molecules. They can also incorporate materials such as radiopaque materials, used to identify the position of the spheres. These emulsions and microspheres can be used in paints and pigments.

Accordingly, in another aspect, the present invention relates to a method of growing anchorage-dependent cells, comprising the steps of:

(a) providing a suspension comprising a polymeric microsphere of the present invention and an inoculum of said cells; and

(b) maintaining said suspension under conditions condusive to cell growth.

In an embodiment, the polymeric microsphere is coated with poly-1-lysine and/or fibronectin. This advantageously helps improves the adhesion of the cells to the polymeric microspheres. In particular, by coating triple layers of fibronectin, poly-1-lysine and fibronectin in a layer-by-layer fashion, poly-1-lysine and fibronectin are bound more tightly on the surface of the microspheres, which acts as foot-holds for cells to adhere. Further, by using the polymeric microspheres of the present invention, wherein the densities of the emulsion is substantially similar, the resultant microspheres have more homogenous surface morphologies (FIG. 5). Advantageously, the consistency of the surface allows the cells to adhere better to the microsphere. As exemplified, this has resulted in at least 70% cell adhesion and high cell viability. Accordingly, in another embodiment, the polymeric microspheres may be coated with one layer of poly-1-lysine. In another embodiment, the polymeric microspheres may be coated with one layer of fibronectin. In another embodiment, the polymeric microspheres may be coated with twice layers of poly-1-lysine and fibronectin in a layer-by-layer process. In other embodiments, the polymeric microspheres comprises two layers of poly-1-lysine on the surface of the microsphere and one layer of fibronectin on the surface of the microsphere, wherein the layer of fibronectin is sandwiched between the two layers of poly-1-lysine.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Certain embodiments of the invention will now be described with reference to the following examples which are intended for the purpose of illustration only and are not intended to limit the scope of the generality hereinbefore described.

### EXAMPLES

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Materials and Chemicals

Two Polycaprolactone (PCL) variants were sourced from Sigma-Aldrich. These are specified by the manufacturer as having average molecular weights of 45 KDa (Cat. No. 704105, referred to as PCL45k), and 80 kDa (Cat. No. 440744, referred to as PCL80k), respectively. Polyvinyl alcohol (PVA, Cat. No. 363170) with Mw 13-23 KDa and 87-89% hydrolyzed, Pluronic F-127 (F-127, Cat. No. P2443), and potassium carbonate (K₂CO₃, Cat. No. 310263) were also sourced from Sigma-Aldrich. Dichloromethane (DCM, ACS grade) was purchased from J.T.Baker (Cat. No. 9324-03), while sodium hydroxide (NaOH, AR grade) was purchased from Goodrich Chemical Enterprise (GCE). Denatured ethanol (99%, Technical grade) was from International Scientific Pte Ltd in Singapore, and absolute ethanol (ACS grade) from Merck. Pure water was delivered from a PURELAB Option Q7 Water Systems, ELGA LabWater. PTFE Tubing (Inner diameter: 0.31 mm) was acquired from Sigma-Aldrich (Cat. No. 58698-U). BD PrecisionGlide™ 30G needles (inner diameter: 0.16 mm; outer diameter: 0.31 mm) were from BD (Becton, Dickinson and Company, REF 305107), and 33G Metal Hub needles (inner diameter: 0.11 mm; outer diameter: 0.21 mm) from Hamilton Company (Cat. No. 91033). Poly-propylene (PP) Syringes with 20 ml volume (REF 4606205V) were sourced from B. Braun Medical Inc, and 50 ml volume syringes (REF 300144) were from Becton, Dickinson and Company. PP tubes of 1.5 ml (Cat. No. 616 201), 15 ml (Cat. No. 188 271) and 50 ml (Cat. No. 210 261) were acquired from Greiner Bio-One GmbH. A Gemini 88 Dual Syringe Rate pump was acquired from KD Scientific Inc. The analytical balance (Model AG204) was purchased from METTLER-TOLEDO(S) PTE LTD. The T 18 digital ULTRA-TURRAX homogenizer was purchased from IKA-Works Company.

General Protocol of Preparing w₁/o Emulsions

1. Weigh 0, 0.252, 3.335, 5.950, 8.930, 10.757 g of K₂CO₃ (MPC) into a 25 ml volumetric flask, then top up to 25 ml with pure water, and obtain the K₂CO₃ solution with the volume (V_{PCS}) of 25 ml, measured in the volumetric flask. The total mass of 25 ml of the K₂CO₃ solution was weighed to be M_{PCS}. Therefore, the density of the K₂CO₃ solution (ρ_{PCS}) can be calculated according to the equation:

ρ_{PCS}=M_{PCS}/V_{PCS}

The concentration of the K₂CO₃ solution can be calculated with the following equation:

$${C}_{P C} = \frac{{M}_{PC}}{{V}_{PCS}}$$

This yields 25 ml of an aqueous K₂CO₃ solution with a density of 1.000, 1.007, 1.106, 1.185, 1.270, 1.320, 1.414, 1.567 g/cm³, respectively, shown in Table 5.

TABLE 5
Density of aqueous K₂CO₃ solutions with
different concentration at 25° C.

| Concentration of | Density of |
|---|---|
| K₂CO₃ | K₂CO₃ |
| solutions | solutions |
| (g/L) | (g/cm³) |
| 0 | 1.000 |
| 10.08 | 1.007 |
| 133.39 | 1.106 |
| 238.00 | 1.185 |
| 357.05 | 1.270 |
| 430.26 | 1.320 |
| 565.64 | 1.414 |
| 830.67 | 1.567 |

2. Weigh 2.0, 3.0, 5.0 g of PCL45k into a 100 ml volumetric flask, then top up to 100 ml with DCM. This yields 100 ml of 50 mg/ml PCL45k/DCM solutions, which all have a density of 1.326 g/cm³. Note: The PCL concentration is preferably within a range from 10 to 250 mg/ml. This will be referred to as the dispersed phase.

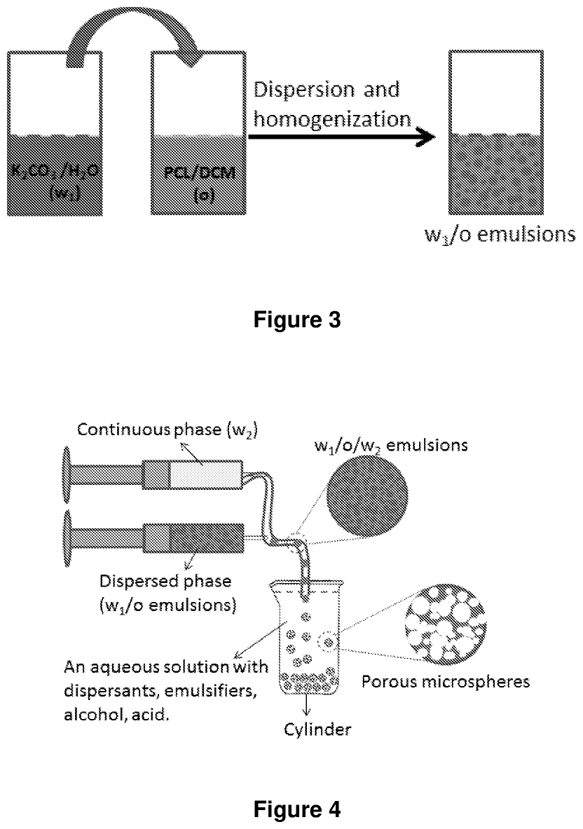

3. Add 10 ml PCL45k/DCM solution into a 50 ml plastic tube and placed it in a T18 emulsifying homogenizer, run at 6,000 rpm, then inject a K₂CO₃ solution into the PCL45k/DCM solution at a flow rate of 0.1 ml/min, for a final volume fraction of K₂CO₃ solution of 10%, 20%, and 40%, respectively. After the K₂CO₃ solution was injected, the mixture was continuously homogenized for 10 min at 9,500 rpm, by the emulsifying homogenizer. Thus, w₁/o emulsions are obtained, as shown in FIG. 3.

General Protocol of Fabricating Porous PCL Microspheres from w₁/o/w₂ Emulsions

1. Weigh 30.0 g of PVA into a 1000 ml glass bottle, then top up to 1000 ml with pure water and shake at 80-90° C., until PVA completely dissolves. This yields 1000 ml of 3 wt.-% aqueous PVA solution. (Note: The PVA concentration range is typically 0.1-5 wt.%.)

2. Weigh 40.0 g of F-127 into a 1000 ml glass bottle, then top up to 1000 ml with pure water and by shake at 80-90° C., until F-127 completely dissolves. This yields 1000 ml of 4 wt.-% aqueous F-127 solution. (Note: The F-127 concentration range is typically 0.1-10 wt.-%.)

3. Mix 25 ml of 3 wt.-% PVA solution with 25 ml of 4 wt.-% F-127 solution, to obtain 50 ml solution: 1.5 wt.-% PVA and 2.0 wt.-% F-127. This is referred to as the continuous phase, i.e., the outer water phase (w₂).

4. Two syringes, one of 20 ml and the other 50 ml, are mounted on syringe pumps, with individually adjustable flow rates. The 50 ml syringe feeds the continuous phase into PTFE tubing (inner diameter: 0.31 mm), with a 30G needle (inner diameter: 0.16 mm; outer diameter: 0.31 mm) used as a connector between the syringe and the tubing. The other 20 ml syringe feeds the dispersed phase into a 33G Metal Hub needle (inner diameter: 0.11 mm; outer diameter: 0.21 mm). As the needle's outer diameter is smaller than the inner diameter of the PTFE tubing, the needle lies (approximately) concentric within the tubing. Thus, the needle injects the dispersed phase into the continuous phase, which is flowing through the tubing surrounding the needle. A schematic diagram of the production method of the w₁/o/w₂ droplets is shown in FIG. 4. The use of other needle sizes (27˜34 gauge) and tubing diameters (Inner diameter: 0.2˜0.8 mm) may similarly be envisaged, depending on the application of this technology and the targeted average droplet diameter.

5. The flow rates of the continuous and dispersed phases are adjusted to generate w₁/o/w₂ droplets. The flow rate of the continuous phase is 100 μl/min, and the flow rate of the dispersed phases is 10 and 20 μl/min, respectively, for preparing porous PCL microspheres and tune the size of the microspheres. (Note: The flow rates of the continuous phase may more generally fall within a range from 1 μl/min to 1000 μl/min, and the flow rates of the dispersed phase may similarly fall within a range from 1 μl/min to 500 μl/min)

6. The formed PCL/DCM droplets are allowed to fall into a column of liquid in a cylinder with height 1000 mm at room temperature, consisting of 700 ml of mixture of an aqueous solution with 0.5% PVA, 0.5% F-127 and ethanol, in which the ethanol volume ratio is 0-80%. The PCL/DCM microdroplets sink by gravity, and their transit through the ethanol/water solution contributes to solidifying them into porous PCL microspheres, by extracting the DCM solvent. They come to rest at the bottom of cylinder. Ethanol is used to accelerate solidification of PCL/DCM droplets. Other solvents, such as isopropyl alcohol (IPA) and methanol, instead of ethanol, may similarly be used to accelerate the solidification of PCL/DCM droplets into porous PCL (pPCL) microspheres. (note: height of the cylinder with water: preferably at least 0.1 meters, with a preferred volume of at least 0.1 litres.)

7. The porous PCL microspheres, collected at the bottom of the cylinder, are allowed to rest for 10-60 min before being transferred into a 15 ml plastic tube. This is followed by three rinses of the microspheres times in pure water to remove PVA and F-127 residue, and twice rinsing in 10-15 ml in denatured ethanol, for 0.5-1 h each, which further extracts and removes DCM residue from the PCL microspheres.

8. Remove the supernatant ethanol, above the precipitated microspheres, and add 10 ml of pure water into the 15 ml tube. This disperses the microspheres in water, which is followed by twice rinsing of the microspheres in pure water.

9. To facilitate probe their inner structure, porous microspheres were sliced using a cryostat (Leica model CM3050 S). Microspheres were dispersed and thus embedded in a clear base mold (OCT PEG matrix, Polyethylene glycol, <5 wt.-%) from Leica Biosystems Richmond, Inc. This suspension was cooled to −30° C., thus solidifying the mold and mounting it onto a disc, placed perpendicular to the knife blade. 70 μm thick sections were sliced and collected in a 50 ml centrifuge tube, at room temperature. This was followed by adding pure water rinsing five times, to dissolve the OCT PEG matrix.

10. Samples of microspheres and sliced microspheres dispersed in water were placed on microscope glass slidesand examined for their morphologies by using a Scanning Electron microscopy: JEOL LV SEM 6360LA, as shown in FIG. 5.

General Protocol for Density and Porosity Measurement of Dried Porous PCL Microspheres

1. After the porous PCL microspheres were rinsed twice in pure water and twice in absolute ethanol. They were then dried in a vacuum oven at room temperature.

2. The mass of 1.5 ml water in a 1.5 ml tube was measured using a calibrated electronic balance, yielding M₁, which is the mass of the water. A mark was placed on the vertical tube in correspondence with the water height.

3. After removing the water and drying the tube, 50-150 mg of PCL microspheres were added into the same tube. The mass of the PCL microspheres was measured in the same manner, yielding M₂.

4. Water was then added into the tube containing the microspheres to the level of the aforementioned mark. The mass of the water was then measured in the same manner, yielding M₃.

5. The volume of the microspheres was derived from: V=(M₁−M₃)/ρ_{w}, where ρ_{w} is the density of water.

6. The density of the dried porous PCL MICROSPHERES was determined from the equation:

Density=mass/volume=M₂/V=M₂ρ_{w}/(M₁−M₃).

The densities are shown in Table 6.

7. The porosity of the porous PCL microspheres can be calculated according to the equation:

Porosity=ρ_{(PCL-d)}/ρ_{(PCL)}.

In the equation, ρ_{(PCL)} is the density of PCL, and ρ_{(PCL-d)} is the density of dry porous PCL microspheres.

TABLE 6
Porosity and density of porous PCL
microspheres from w₁/o/w₂ droplets.

| Volume | | | |
|---|---|---|---|
| fraction | | | |
| of the K₂CO₃ | | | |
| solution in the | 0% | 10% | 15% |
| w₁/o emulsions | ethanol | ethanol | ethanol |
| 10% | 71%* | | |
| | (0.336^{†}, 1.042^{‡}) | | |
| 20% | | | 76%* |
| | | | (0.276^{†}, 1.035^{‡}) |
| 40% | 72%* | 71%* | |
| | (0.323^{†}, 1.04^{‡}) | (0.329^{†}, 1.045^{‡}) | |

The w₁ phase is an aqueous K₂CO₃ solution with a density of 1.32 g/cm³. The oil phase is a 50 mg/ml PCL45k/DCM solution. The w₂ phase is a mixture of 1.5% PVA and 2% F-127 aqueous solutions. The w₁/o/w₂ droplets are collected and solidified in an aqueous solution with 1.5% PVA, 2.0% F-127, and 0-15% ethanol.

* The porosity, † dried density, ‡ wet density (dispersed in water) of the porous microspheres.

Calculation of the Density of the Wet Porous PCL Microspheres in the Water

The density of the wet porous PCL microspheres in water is calculated according to the following equation:

$${ρ}_{PCL \text{-} w} = {ρ}_{PCL \text{-} d} + {ρ}_{w} ( 1 - \frac{{ρ}_{PCL \text{-} d}}{{ρ}_{P C L}} ) \qquad ( 1 )$$

where ρ_{PCL-w} is the density of the wet porous PCL microspheres in water. The densities of the wet porous PCL microspheres are shown in Table 6.

### Example 1

### Fabrication of w₁/o Emulsions

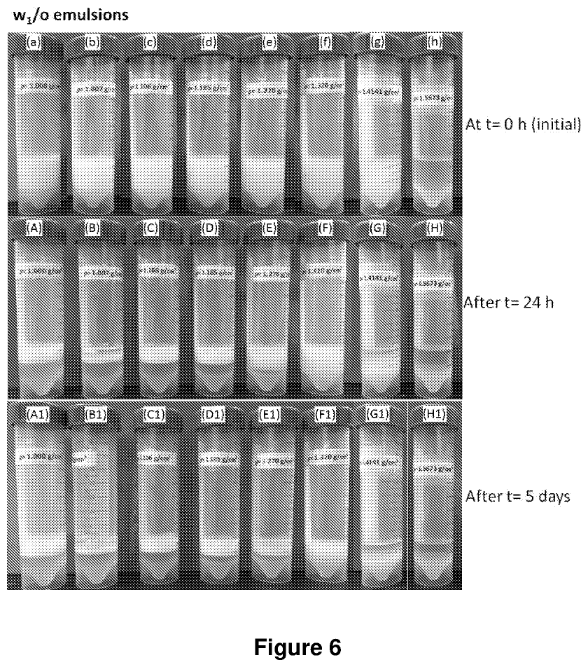

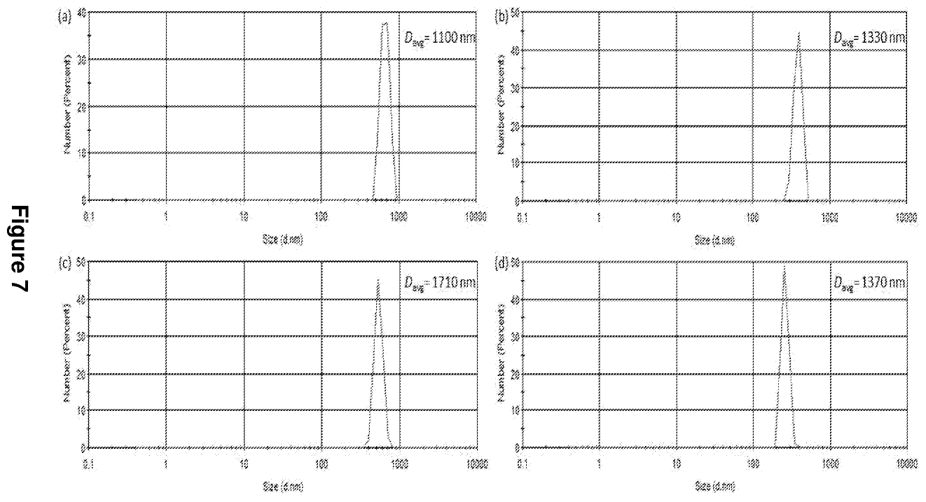

FIG. 3 shows a schematic of the production method for w₁/o emulsions. In a homogenizer, the inner water phase (w₁) of aqueous K₂CO₃ solution (with tunable density) was added into the oil phase (o) of PCL/DCM solutions, to make a w₁/o emulsion. After being homogenized, the w₁/o emulsions are depicted in FIG. 6(a-f).

FIG. 6 shows a bottle test experiment demonstrating the stability, phase separation and upward floating of water droplets in water-in-oil (w₁/o)emulsions at different time intervals of sample preparation: (a-h) 0 h (initial), (A-H) after 24 h and (A1-H1) after 5 days of emulsion preparation. The inner water phase (dispersed phase), with a volume fraction of 20%, is an aqueous K₂CO₃ solution with a density of (a, A, A1) 1.000 g/cm³, (b, B, B1) 1.007 g/cm³, (c, C, C1) 1.106 g/cm³, (d, D, D1), 1.185 g/cm³, (e, E, E1) 1.270 g/cm³, (f, F, F1) 1.320 g/cm³, (g, G. G1) 1.414 g/cm³, and (h, H, H1) 1.567 g/cm³. The oil phase (continuous phase) is 50 mg/ml PCL45k/DCM solution, with a density of 1.326 g/cm³. Emulsions in (f, F, F1) appear stable. Emulsions In (a-e) and are less stable. In (A-E) and (A1-E1), the separated lower phase is the oil phase of PCL45k/DCM solutions. However, in (G-H) and (G1-H1), the separated lower phase is the water phase of aqueous K₂CO₃ solutions.

These solutions of w₁/o emulsions were then allowed to stand for one day, which led to (gravitational) phase separation, as shown in FIG. 6(A-E). The w₁/o emulsions, from the aqueous K₂CO₃ solution with a density of 1.320 g/cm³, which matches the density of PCL/DCM solution (1.326 g/cm³), maintains its stability and does not show appreciable phase separation (FIG. 6F). This suggests that the stable w1/o emulsions can be fabricated with a water phase that has a matched density with the oil phase.

According to FIG. 6, For PCL/DCM and K₂CO₃ aqueous solution, the w/o emulsions may remain stable for 5 days when the alteration of their density differential is no more than 0.015 g/ml. It is expected that the w/o emulsions, of K₂CO₃ aqueous solution in PCL/DCM solution, when substantially density matched and prepared at 25° C., may also remain stable for about 5 days when temperature ranges from about 10° C. to about 30° C.

FIG. 7 shows the average size and size distribution of water-in-oil (w/o) emulsions, measured on Malvern Zetasizer NaNo ZS90 after the emulsions being prepared for 2 h. The inner water phase (dispersed phase) is an aqueous K₂CO₃ solution with a density of 1.320 g/cm³. The oil phase (continuous phase) is 50 mg/ml PCL45k/DCM solution, with a density of 1.326 g/cm³. In the w/o emulsions, the volume fraction of the water phase is (a) 2.5%, (b) 5.0%, (c) 10%, and (d) 20%.

### Example 2

### Fabrication of Porous PCL Microspheres by Using w₁/o/w₂ Droplets

The w₁/o/w₂ droplets can be made, with the stable w₁/o emulsions (for example: FIG. 6f) as the dispersed phase and the aqueous solution of a dispersant or an emulsifier (for example: PVA) as the continuous phase (w₂), by using the microfluidic device, as shown in FIG. 4. The w₁/o/w₂ droplets were collected and gravitationally sank into a cylinder with a large volume of aqueous solution containing ethanol, dispersants or/and emulsifying additives. The droplets will be solidified to be porous microspheres during the gravitationally sinking. FIG. 5 shows the porous PCL microspheres. These porous microspheres have a high porosity, and low density, as shown by the data in Table 6. Across a range of fractions (10-40%), for the water emulsion, and a range of ethanol solution concentrations, the solidified microsphere porosity remains consistent, at 71-76%.

FIG. 5 illustrates SEM images of uniform-sized porous PCL microspheres from w₁/o/w₂ droplets by microfluidics. Spheres were formed from a mixture of 1.5% PVA and 2% F-127 aqueous solutions as the continuous phase (w₂) and the w₁/o emulsion solution as the dispersed phase, with flow rates: V_{c}=100 μl/min and V_{d}=20 μl/min, respectively. In w₁/o emulsions, the w₁ phase is the aqueous K₂CO₃ solution (its density: 1.32 g/cm³), and the oil phase (o) is a 50 mg/ml PCL/DCM solution. In the w₁/o emulsions, the volume fraction of the w₁ phase is (a-d) 10% and (e, f) 20%, respectively. The w₁/o/w₂ droplets were collected in (a, b) an aqueous solution containing 0.5% PVA, 0.2% F-127; (c-f) an aqueous solution containing 0.5% PVA, 0.2% F-127, and 15% ethanol. Cross-section of microspheres in images (b, d, f) are the corresponding porous microspheres in images (a, c, e), respectively. The diameters of the porous PCL microsphere are (a, b) 197±18 μm, (c, d) 176±18 μm, (e, f) 160±10 μm.

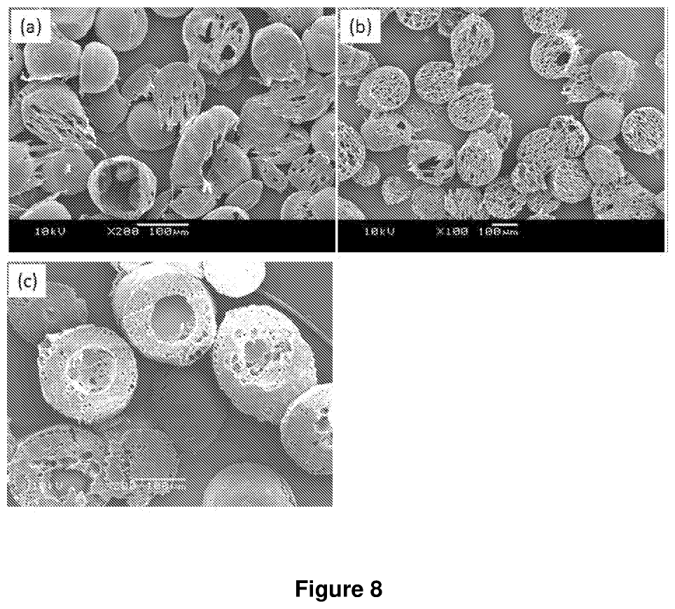

FIG. 8 shows SEM images of cross-section of porous PCL microspheres obtained from w₁/o/w₂ double emulsion. Spheres were formed from a mixture of 1.5% PVA and 2% F-127 aqueous solutions as the continuous phase (w₂) and the w₁/o emulsion solution as the dispersed phase, with flow rates: Vc=100 μl/min and Vd=10 μl/min, respectively. In w₁/o emulsions, the w₁ phase is the aqueous K₂CO₃ solution (density: 1.32 g/cm³), and the oil phase (o) is a 50 mg/ml PCL/DCM solution. In the w₁/o emulsions, the volume fraction of the w₁ phase is 10%. The w₁/o/w₂ droplets were collected in an aqueous solution containing 0.5% PVA, 0.2% F-127, and (a) 0 vol %, (b) 15 vol % and (c) 80 vol % ethanol, respectively. Some microspheres have a porous core-shell structure, with the shell thickness of about 0.1-65 μm.

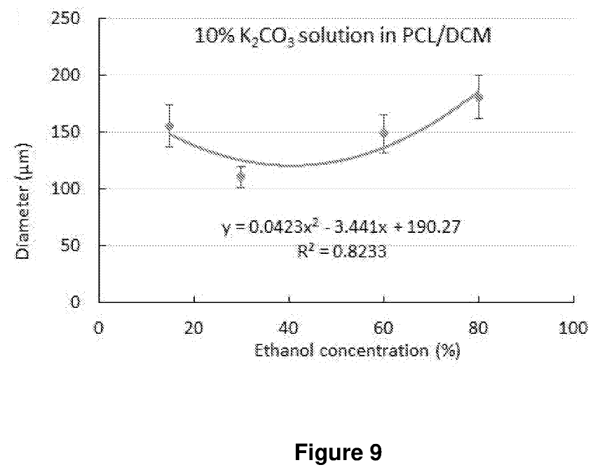

FIG. 9 illustrates the distribution of porous PCL microspheres obtained from w₁/o/w₂ double emulsion. Spheres were formed from a mixture of 1.5% PVA and 2% F-127 aqueous solutions as the continuous phase (w₂) and the w₁/o emulsion solution as the dispersed phase, with flow rates: Vc=100 μl/min and Vd=10 μl/min, respectively. The volume fraction of the w₁ phase is 10%.

### Example 3

### Fabrication of w₁/o Emulsions (Silicone Oil)

Silicone oils can be used as oil phase in these emulsions, applied in cosmetics. Water-in-silicone oil emulsions can become much more stable.

Materials and Chemicals:

Silicone oil AP100 (Cat. No. 10838, ρ_{AP100}=1.06 g/cm³) and silicone oil AP1000 (Cat. No. 10842, ρ_{AP1000}=1.09 g/cm³), as well as sodium chloride (NaCl, Cat. No. S7653) purchased from Sigma-Aldrich (Singapore).

Protocol of Preparing w/o Emulsions:

1. Weigh 0, 2.492 and 3.533 g of NaCl (M_{SC}) into a 25 ml volumetric flask, respectively, then top up to 25 ml with pure water, to obtain NaCl solution with a volume (V_{SCS}) of 25 ml, measured in a volumetric flask. The total mass of 25 ml of the NaCl solution was weighed to be M_{SCS}. Therefore, the density of the NaCl solution (ρ_{SCS}) can be calculated according to the equation:

ρ_{SCS}=M_{SCS}/V_{SCS}.

The concentration of the NaCl solution can be calculated with the following equation:

C_{SC}=M_{SC}/V_{SCS}.

This yields 25 ml of an aqueous NaCl solution with a density of 0, 1.061 and 1.086 g/cm³, respectively.

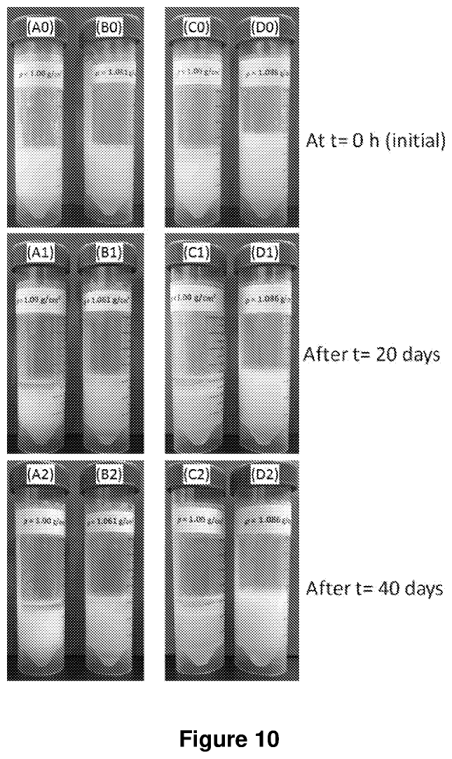

2. Add 10 ml silicone oil AP100 with ρ_{AP100}=1.06 g/cm³ (or silicone oil AP1000 with ρ_{AP1000}=1.09 g/cm³) into a 50 ml plastic tube, respectively, and placed in a T18 emulsifying homogenizer, run at 9,000 rpm, then inject water with a density of 1.00 g/cm³ and a NaCl solution with ρ_{SCS}=1.061 g/cm³ (or ρ_{SCS}=1.086 g/cm³) into silicone oil, respectively, at a flow rate of 0.1 ml/min, for a final volume fraction of NaCl solution of 20%. After the NaCl solution was injected, the mixture was continuously homogenized for 10 min at 11,000 rpm, by the emulsifying homogenizer. Thus, w/o emulsions are obtained, as shown in FIG. 10.

FIG. 10 shows the results of bottle test experiments illustrating the stability, phase separation and upward floating of water droplets in w/o emulsions at different time intervals of sample preparation. The inner water phase (w) is an aqueous NaCl solution (with tunable density) and the oil phase (o) is silicone oil. The inner water phase (dispersed phase), with a volume fraction of 20%, is pure water (A0, A1, A2, C0, C1, C2) with a density of 1.000 g/cm³, and a aqueous NaCl solution with a density of (B, B1, B2) 1.061 g/cm³ and (C0, C1, C2) 1.086 g/cm³. The oil phase (continuous phase) is silicone oil, with a density of 1.06 g/cm³ (A0, B0, A1, B1, A2, B2) and 1.09 g/cm³ (C0, D0, C1, D1, C2, D2). These solutions of w/o emulsions, with pure water in silicone oil were then allowed to stand for 20 and 40 days, which led to (gravitational) phase separation, as shown in FIG. 10 (A0, A1, A2) and FIG. 10 (C0, C1, C2). The w/o emulsions, from the aqueous NaCl solution with a density of 1.061 and 1.086 g/cm³, which matches the density of silicone oil with a density of 1.06 and 1.09 g/cm³, respectively, maintains its stability and does not show appreciable phase separation, as shown in FIG. 10 (B, B1, B2) and FIG. 10 (D0, D1, D2), respectively.

According to FIG. 10, for silicone oil and NaCl aqueous solution, the emulsion may remain stable when the alteration of their density differential is no more than 0.015 g/ml and when temperature variation is no more than 50° C. It is expected that the w/o emulsions, of NaCl aqueous solution in silicone oil, when substantially density matched and prepared at 25° C., may remain stable for about 40 days when temperature ranges from about 0° C. to about 70° C.

### Example 4

### Coating Porous PCL Microspheres with Poly-1-Lysine and Fibronectin

Poly-1-lysine hydrobromide (PLL, Mw70,000-150,000; Cat. No. P6282) and Fibronectin (FN; Cat. No. F0895) were bought from Sigma-Aldirch.

The sterilized porous PCL45K microspheres were coated with multiple layers of PLL and FN by incubating 150 mg of porous PCL microspheres in 1 ml PBS solution of 120 ng/ml of FN in a 1.5 ml Eppendorf tube for 15 hours at room temperature. After being coated with one layer of FN, the coated PCL microspheres were rinsed twice with PBS. Then the coated PCL were incubated again into 1 ml PBS solution of 240 ng/ml of PLL in a 1.5 ml Eppendorf tube for 15 hours at room temperature, followed by being rinsed twice with PBS. Then PLL+FN-coated PCL microspheres were incubated once again into 1 ml PBS solution of 120 μg/ml of FN in a 1.5 ml Eppendorf for 15 hours at room temperature, as the outer protein layer on PCL microspheres. Thereafter, the coated PCL microspheres were rinsed twice with PBS, and stored at 4° C. before used, as designated as pPCL-FN+PLL+FN microspheres, which can be used as microcarriers (MCs) to support stem cell culture.

### Example 5

### Growth of Human Mesenchymal Stem Cells (hMSCs) on PCL Microspheres

Reagents and Chemicals:

High glucose Dulbecco's modified Eagle's medium (DMEM; Cat. No. 11960), fetal bovine serum (FBS; Cat. No. 10270), 0.25% trypsin-EDTA (Cat. No. 25200) and Dulbecco's phosphate buffered saline (PBS; Cat. No. 14190) were purchased from Gibco, USA. Bovine Serum Albumin (BSA; A3156) was purchased from Sigma, St. Louis, MO. Erlenmeyer Flask (Cat. No. 431404) were purchased from Corning (Corning, N.Y.).

MSC Culture:

Human Wharton's jelly MSCs (WJ-1) was purchased from PromoCell (Heidelberg, Germany) MSCs were cultured in all) medium, consisting alpha Minimum Essential Medium (αMEM), supplemented with 10% (v/v) FBS, and maintained in a 37° C. 5% CO₂ humidified incubator. Single-cell suspensions of hMSCs were prepared by trypsinization by 0.25% trypsin-EDTA. MSC lines were used in microcarrier (MC) expansion experiments at passage 6-10.

Propagation of hMSCs on LPCL−FN+PLL+FN in shake flask cultures:

One-hundred-and-twenty-five milliliter Erlenmeyer flask, containing 150 mg of LPCL−FN+PLL+FN microspheres in 10 ml of α10 medium were seeded with 4.8×104 cells/ml of WJ1. The culture was maintained for up to 7 days in agitated shake flasks. Cell density was determined on day 7 using NucleoCounter NC-3000 (Chemometec, Allerod, Denmark).

Immunophenotypic Analysis:

Multipotency of hMSCs was measured on day 7 using flow cytometry. Briefly, MSCs were harvested using 0.25% Trypsin-EDTA, filtered through a 40-nm sieve (Cat No. #352340; BD Biosciences, San Jose, Calif.), then incubated with mouse primary antibodies (Biolegend, San Diego, Calif.) CD34 (1:10), CD73 (1:10), CD90 (1:10), CD105 (1:20), and CD146 (1:50) for 15 min The cells were washed in 1% BSA/PBS, incubated for 15 min incubation with 1:500 goat anti-mouse FITC-conjugated antibody (Cat No. #P0161; DAKO, Santa Clara, Calif.). Cells were washed and resuspended with 1% BSA/PBS for analysis using a Guava EasyCyte flow cytometer running Guava ExpressPlus software (Guava Technologies, Merck, Darmstadt Germany) Gates were typically set at the point of intersection between the negative and the positive stains, after which the percentage of cells from the negative control within the gate was subtracted from the positive. Cells stained with secondary antibody alone were used as negative control.

Attachment and Expansion of hMSC:

FIG. 11 illustrates photographs of hMSCs performance on porous PCL45k microspheres with a density of 1.035 g/cm³ under agitation culture. The pPCL45k microspheres, coated with three layers of FN+PLL+FN, were from FIG. 3(e).

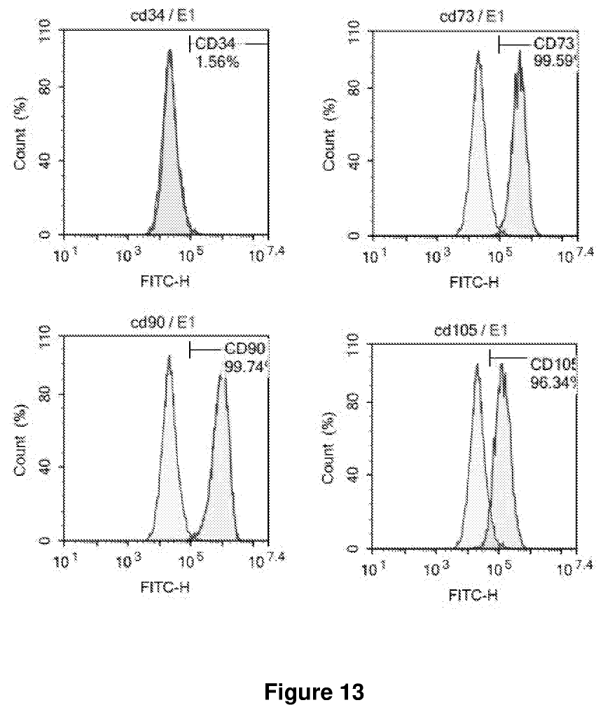

For hMSCs on pPCL45−FN+PLL+FN microspheres from FIG. 5e, with density of 1.035 g/cm³, fabricated from w/o emulsions with 20% aqueous K₂CO₃ solution and 50 mg/ml PCL45k/ml solution and collected in 15% ethanol, cell attachment during the first 2 hours is 73±1.4% (FIG. 11a). Cell density following 7 days' expansion under agitation is 5.5×10⁵ cells/ml. Cells have a ˜11-fold expansion and the cell grew mainly as cells/MC aggregates (FIG. 11b). hMSC, cultured on pPCL45−FN+PLL+FN microspheres from FIG. 11, retained their characteristic immunophenotype, as illustrated by low expression (<5%) of marker CD34, and high expression of CD73, CD90 and CD105 markers, as shown in FIG. 13.

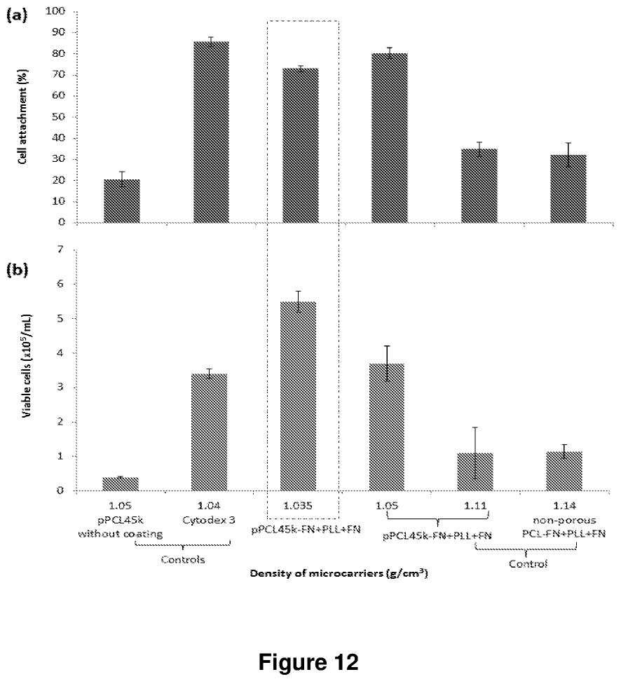

FIG. 12 shows hMSC performance on pPCL45k MCs with low and high density under agitation culture. Graph (a) demonstrates the percentage of cell attachment on the MCs after being seeded for 2 h, and graph (b) demonstrates cell density following 7 days' expansion. The pPCL45k MCs (within the red dash line)) from FIG. 11, with density of 1.035 g/cm³, coated with three layers of FN+PLL+FN, were fabricated from w/o emulsions with 20% aqueous K₂CO₃ solution and 50 mg/ml PCL45k/ml solution. The formed droplets were subsequently exposed to in 15% ethanol, to solidify them. As controls, pPCL45k MCs were fabricated from 50 and 170 mg/ml PCL45k/DCM solutions, with Q_{d}=5 μl/min and Q_{c}=100 μl/min, solidified in 90% ethanol. These microcarriers have a measured density of 1.05 and 1.11 g/cm³, respectively. Non-porous MCs were fabricated from 30 mg/ml PCL45k/DCM, with Q_{d}=100 μl/min and Q_{c}=300 μl/min. The droplets were solidified by evaporation (i.e. not exposed to ethanol), and have a measured density of 1.14 g/cm³. Cytodex 3, pPCL45k (Density: 1.05 and 1.11 g/cm³), and non-porous PCL−FN+PLL+FN MCs, coated with three layers of FN+PLL+FN, are used as positive control; uncoated pPCL45k PCL MCs are used as a negative control.

FIG. 12a shows that there is no significant difference in attachment efficiency, when comparing Cytodex 3 (benchmark, commercial) microcarriers with pPCL+FN+PLL+FN microcarriers, having a density of 1.035 and 1.05 g/cm³, which promote cell attachment above 70%, higher than those on pPCL+FN+PLL+FN microcarriers with a density of 1.1 g/cm³ and on non-porous PCL+FN+PLL+FN microcarriers, as well as the negative control, of uncoated pPCL microcarriers.

FIG. 12b shows that hMSCs grown on pPCL45−FN+PLL+FN microspheres from FIG. 6e, with density of 1.035 g/cm³, have higher fold-expansion than on other MCs, including Cytodex 3 (˜8.5-fold expansion), pPCL+FN+PLL+FN MCs with density of 1.05 g/cm³ (˜9-fold expansion), non-porous PCL+FN+PLL+FN MCs (˜3-fold expansion).

1. An emulsion comprising:
a) a water phase, the water phase is an aqueous solution comprising a salt; and
b) an oil phase, the oil phase comprising a polymer;
wherein the oil phase is immiscible with the water phase, and
wherein the density differential of the water phase and oil phase is less than about 0.02 g/cm³.

2. The emulsion according to claim 1, the emulsion is stable for at least 5 days.

3. The emulsion according to claim 1 or 2, wherein the densities of the water phase and oil phase are independently from about 1 g/cm³ to about 1.7 g/cm³.

4. The emulsion according to any of claims 1 to 3, wherein the polymer has a concentration from about 10 mg/mL to about 250 mg/mL.

5. The emulsion according to any of claims 1 to 4, wherein the polymer is selected from polycaprolactone (PCL), polylactic acid (PLA), polyglycolide (PGA), poly(lactic-co-glycolic acid) (PLGA), poly(glycolide-co-caprolactone), poly (glycolide-co-trimethylene carbonate), polystyrene (PS), polyethylene terephthalate (PET) or their copolymers thereof.

6. The emulsion according to any of claims 1 to 5, wherein the salt is selected from potassium carbonate (K₂CO₃), potassium acetate (K(CH₃CO₂)), potassium bromide (KBr), potassium chloride (KCl), potassium iodide (KI), potassium bisulfate (KHSO₄), potassium sulfate (K₂SO₄), potassium phosphate monobasic (KH₂PO₄), calcium chloride (CaCl₂), calcium acetate (Ca(C₂H₃O₂)₂), zinc chloride (ZnCl₂), zinc sulfate (ZnSO₄), sodium acetate (Na(CH₃CO₂)), sodium carbonate (Na₂CO₃), sodium bromide (NaBr), sodium chloride (NaCl), sodium iodide (NaCI), sodium bisulfate (NaHSO₄), sodium sulfate (Na₂SO₄), sodium thiosulfate (sodium hyposulfite) (Nu₂S₂O₃), sodium sulfite (Na₂SO₃), sodium phosphate tribasic (Na₃PO₄), sodium phosphate dibasic (Na₂HPO₄), sodium perchlorate (NaClO₄), ammonium acetate (CH₃COONH₄), ammonium carbonate ((NH₄)₂CO₃), ammonium sulfate ((NH₄)₂SO₄), lithium chloride (LiCl), magnesium chloride (MgCl₂), magnesium sulfate (MgSO₄), silver nitrate (AgNO₃), cupric sulfate (CuSO₄), cesium sulfate (Cs₂SO₄), cesium chloride (CsCl) or cobaltous chloride (CoCl₂).

7. The emulsion according to any of claims 1 to 6, wherein the oil phase further comprises dichloromethane.

8. The emulsion according to any of claims 1 to 7, comprising:
a) the water phase at less than 50 vol % of the final emulsion volume; and
b) the oil phase at more than 50 vol % of the final emulsion volume.

9. The emulsion according to any of claims 1 to 8, the emulsion does not have an emulsifier.

10. The emulsion according to any of claims 1 to 9, further comprising an outer continuous phase to form a double emulsion.

11. The emulsion according to claim 10, wherein the outer continuous phase comprises a dispersant from about 0.1 wt % to about 10 wt % of the outer continuous phase and an emulsifier from about 0.1 wt % to about 10 wt % of the outer continuous phase.

12. The emulsion according to claim 10 or 11, wherein the outer continuous phase is a outer water continuous phase.

13. A method of making an emulsion, comprising the steps of:
a) homogenising a water phase and an oil phase to form an emulsion, the water phase is an aqueous solution comprising a salt, the oil phase comprising a polymer;
wherein the oil phase is immiscible with the water phase, and
wherein the density differential of the water phase and oil phase is less than about 0.02 g/cm³.

14. The method according to claim 13, further comprising the step of flowing the emulsion into an outer continuous phase to form a double emulsion.

15. The method according to claim 14, wherein the outer continuous phase comprises a dispersant from about 0.1 wt % to about 10 wt % of the outer continuous phase and an emulsifier from about 0.1 wt % to about 10 wt % of the outer continuous phase.

16. A method of forming a polymeric microsphere, comprising the steps of:
a) homogenising a water phase and an oil phase to form an emulsion, the water phase is an aqueous solution comprising a salt, the oil phase comprising a polymer, wherein the oil phase is immiscible with the water phase, and wherein the density differential of the water phase and oil phase is less than about 0.02 g/cm³;
b) flowing the emulsion into an outer continuous phase to form a double emulsion, the outer continuous phase comprising a dispersant from about 0.1 wt % to about 10 wt % of the outer continuous phase and an emulsifier from about 0.1 wt % to about 10 wt % of the outer continuous phase; and
c) immersing the double emulsion in a solvent exchange liquid to form the polymeric microsphere.

17. A polymeric microsphere obtained according to the method of claim 16.

18. A polymeric microsphere according to claim 17, the microsphere having a porosity of about 10% to about 90% and wherein the variance of the microsphere particle size distribution is less than about 0.8.

19. The polymeric microsphere according to claim 17 or 18, the microsphere having a particle size of about 100 μm to about 500 μm.

20. Use of the polymeric microsphere according to any of claims 17 to 19 as microcarriers for cell culture.
